(19) United States
(12) Patent Application Publication
(10) Pub. No.: US 2007/0154464 A1
(43) Pub. Date: Jul. 5, 2007

(54) Methods for screening for agents capable of modulating t lymphocyte function in response to a herpes simplex virus-infected cell

(76) Inventors: Keith R. Jerome, Seattle, WA (US); Derek D. Sloan, Seattle, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/595,840

(22) PCT Filed: Nov. 15, 2004
(86) PCT No.: PCT/US04/38050

§ 371 Date: May 15, 2006

Related U.S. Application Data
(60) Provisional application No. 60/520,136, filed on Nov. 14, 2003.

(51) Int. Cl. A61K 48/00; C12Q 1/70; A61K 39/395

(52) U.S. Cl. 424/93.21; 435/5; 424/144.1

(57) ABSTRACT

Disclosed are methods for screening an agent for activity in modulating a T lymphocyte. The methods generally comprises contacting a HSV U_{S}3-expressing cell with a T lymphocyte in the presence or absence of the agent and monitoring a physiological change associated with CTL function to determine whether the agent modulates CTL activity. In particular, the HSV U_{S}3-expressing cell line can be a HSV-infected fibroblast and the physiological change associated with CTL function is a reduction or inhibition of the phosphorylation of HSP90 and/or a 50 kD CTL protein. Also disclosed are methods for blocking suppression of CTL activity against HSV-infected target cells, as well as methods for suppressing CTL activity against a target antigen.

### CROSS-REFERENCES TO RELATED APPLICATIONS

The present application claims benefit to U.S. Provisional Application No. 60/520,136, filed Nov. 14, 2003, the entire disclosure of which is incorporated herein by reference.

### STATEMENT AS TO RIGHTS TO INVENTIONS MADE UNDER FEDERALLY SPONSORED RESEARCH AND DEVELOPMENT

This work was supported in part by grant ROI-A147378 from the National Institutes of Health. The U.S. Government may have certain rights in the invention.

### BACKGROUND OF THE INVENTION

Cytotoxic T lymphocytes (CTL) play a pivotal role in a variety of human illnesses. For example, CTL are needed to control the acute and chronic stages of most viral infections including, for example, HIV, hepatitis B virus, and the herpes viruses (e.g., herpes simplex virus (HSV), cytomegalovirus (CMV), and Ebstein-Barr virus (EBV)). (See, e.g., Ogg et al, Science 279:2103, 1998; Pantaleo et al., Nature 370:463, 1994; Guidotti et al., Immunity 4:25, 1996; Mueller et al, J. Exp. Med. 195:651, 2002; Khan et al., J. Immunol. 169:1984, 2002; Heslop et al., Nat. Med. 2:551, 1996). CTL are also believed to play a role in cancer surveillance, and CTL clones raised against tumor antigens have been used successfully in adoptive immunotherapy regimens. (See, e.g., Amrolia et al., Blood 12:12, 2002; Seki et al., J. Immunol. 168:3484, 2002; Titu et al., Cancer Immunol. Immunother. 51:235, 2002; Romero et al., Immunol. Rev. 188:81, 2002). However, CTL can cause life-threatening illnesses such as transplant rejection, graft-versus-host disease, and autoimmunity. (See, e.g., Barry and Bleackley, Nat. Rev. Immunol. 2:401, 2002; Burrows et al., J. Exp. Med. 179:1155, 1994; Valujskikh et al., Nat. Immunol. 3:844, 2002). Thus, CTL are involved in both preventing and promoting pathogenesis.

CTL are a critical component of many successful immune responses, but they have the potential to injure the host. To prevent inappropriate and excessive lymphocyte responses, T cells have evolved strategies to downregulate effector functions (Horwitz and Sarvetnick, Immunol. Rev. 161:241, 1999; Smyth et al., Nat. Immunol. 2:293, 2001; Parojs and Abbas, Science 280:243, 1998; Walker and Abbas, Nat. Rev. Immunol 2:11, 2002). Therefore, their activity is closely regulated by multiple receptor-ligand interactions. Before they can function as effectors, CTL must be activated by antigen presenting cells and triggered by target cells. Once activated, the immune system has mechanisms to control a CTL response and prevent the destruction of healthy host cells. Activated CTL can be depleted through Fas-mediated apoptosis, a process called activation-induced cell death (AICD). Alternatively, activated CTL can become anergic or differentiate into a functionally impaired, non-responsive, or memory-like state. (See, e.g., Bergmann et al., J. Immunol. 163:3379, 1999; Lukacher et al., J. Immunol. 163:3369, 1999; Tharn et al., J. Immunol. 168:1190, 2002)). Microbes, in particular viruses that have co-evolved along with their hosts, appear to have pirated several of these strategies in order to manipulate and consequently evade the host immune response. Many viral homologues of immune modulatory cellular ligands, signaling proteins, chemokines and cytokines have been shown to inhibit lymphocyte function. (Spriggs, Ann. Rev. Immunol. 14:101, 1996; Tortorella et al., Ann. Rev. Immunol. 18:861, 2000).

Because CTL are involved in controlling the acute, lytic phase and persistent, latent phase of HSV infection, HSV has evolved numerous mechanisms to evade CTL as part of its survival strategy. HSV-infected cells can avoid CTL detection by interfering with TAP-mediated peptide loading onto MHC. (See Fruh et al., Nature 375:415, 1995; Hill et al., Nature 375:411, 1995; Koelle et al., J. Clin. Invest. 91:961, 1993). Furthermore, several HSV genes have been shown to inhibit CTL-induced apoptosis of HSV-infected target cells. (See Jerome et al., J. Immunol. 167:3928, 2001; Jerome et al., Arch. Virol. 146:2219, 2001; Jerome et al., J. Virol. 73:8950, 1999; Jerome et al., J. Virol. 72:436, 1998; Aubert and Blaho, Microbes Infect. 3:859, 2001). An alternative mechanism described nearly a decade ago, demonstrated that HSV-infected cells were capable of inhibiting the lytic function of various immune effector cells. (Confer et al., Proc. Natl. Acad. Sci. USA 87:3609, 1990; Posavad and Rosenthal, J. Virol. 66:6264, 1992; Posavad et al., J. Immunol. 151:4865, 1993). In these models, when effector cells were incubated with HSV-infected fibroblasts, they lost the ability to lyse subsequently added target cells. Subsequent studies focused on the cell-to-cell spread of HSV to effector cells. (See Posavad et al., J. Virol. 68:4072, 1994; York et al., Cell 77:525, 1994). In addition, it was reported that HSV-infected CTL expressed increased levels of Fas ligand and thus could be silenced through fratricide-induced apoptosis. (See Raftery et al., J. Exp. Med. 190:1103, 1999).

The ability to specifically modulate CTL function is an attractive therapy concept. However, many of the molecular pathways and targets for CTL manipulation remain to be identified, including, for example, the molecular pathways involved in viral (e.g., HSV) evasion of CTL lytic function. Thus, there is a need in the art for agents and methods of effectively modulating CTL activity by targeting novel molecular pathways, as well as a need for methods of identifying these agents. Such agents and methods would be useful for, for example, in stimulating dysfunctional CTL (such as, e.g., those that exist in persistent viral infections and tumor states) or, alternatively, to promote immune suppression (such as, e.g., in autoimmune disease). The present invention as described herein meets these needs and more.

### BRIEF SUMMARY OF THE INVENTION

The present invention provides methods for screening an agent for activity in modulating T lymphocyte function generally including the following steps: (1) contacting the agent with (a) a cell expressing a HSV U_{S}3 polypeptide and late HSV proteins; and/or (b) a T lymphocyte; (2) contacting the HSV U_{S}3-expressing cell with the T lymphocyte; (3) contacting a second HSV U_{S}3-expressing cell with a second T lymphocyte, wherein the second HSV U_{S}3-expressing cell and the second T lymphocyte are not contacted with the agent; (4) determining for each of the first and second T lymphocytes the level of a physiological change associated with T lymphocyte function; and (5) comparing the relative levels of the physiological change determined for each of the first and second T lymphocytes to determine whether the agent modulates T lymphocyte function. Typically, the T lymphocyte is a cytotoxic T lymphocyte (CTL) and the T lymphocyte function is a (CTL) function. In certain embodiments, the method further includes contacting the first and second T lymphocytes with a second agent that activates the T cell receptor (a “TcR-activating agent”).

In certain embodiments, the cell expressing the HSV U_{S}3 polypeptide and late HSV proteins is a fibroblast. Further, the cell expressing the HSV U_{S}3 polypeptide and late HSV proteins can be, for example, a cell (for example, a fibroblast) infected with a herpes simplex virus (HSV) (e.g., HSV-1 or HSV-2), or a recombinant cell. In some embodiments, the HSV U_{S}3 polypeptide has an amino acid sequence that has 90% sequence identity with the sequence set forth in SEQ ID NO:1 or SEQ ID NO:2. In one specific embodiment, the HSV U_{S}3 polypeptide has the amino acid sequence set forth in SEQ ID NO:1 or SEQ ID NO:2.

In certain embodiments that include contacting the first and second CTLs with a TcR-activating agent, the TcR-activating agent can be, for example, staphylococcal enterotoxin B (SEB) or an anti-CD3 antibody (e.g., a solid phase anti-CD3 antibody). In some embodiments utilizing an anti-CD3 antibody as the TcR-activating agent, the CTL is also contacted with a target cell expressing an Fcγ receptor (e.g., a FcγR⁺ P815 target cell).

In yet other embodiments, the TcR-activating agent is a target antigen bound to an MHC class I molecule on the surface of a target cell, where the first and second CTL specifically recognize the target antigen. The target cell can be, for example, an EBV-transformed B cell line. Further, in certain embodiments, the HSV U_{S}3-expressing cell and the target cell are the same cell.

The T lymphocyte physiological change measured can be, for example, production of a cytokine (e.g., IFN-γ or TNF-α), exocytosis of lytic granules from a CTL, a change in the phosphorylation state of a cellular protein (for example, a CTL protein such, e.g. heat shock protein 90 (HSP90) or other protein associated with the TcR or the TcR signaling cascade, (such as a 50 kD CTL protein)), or a change associated with apoptosis of the target cell. The apoptosis-associated physiological change can be, for example, lysis of the target cell (e.g., measure by detecting release of ⁵¹Cr from a ⁵¹Cr-labeled target cell), activation of a caspase (e.g., caspase 3).

In a specific embodiment of the present invention, the method comprises the following steps: (1) contacting the agent with (a) a HSV-infected cell; and/or (b) a CTL; (2) contacting the HSV-infected cell with the CTL; (3) contacting a HSV-infected cell with a second CTL, wherein the HSV-infected cell and the second CTL are not contacted with the agent; (4) determining for each of the first and second CTL the level of phosphorylation of HSP90 and/or a 50 kD CTL protein; and (5) comparing the relative levels of the phosphorylation of HSP90 and the 50 kD CTL protein for each of the first and second CTL to determine the agent reduces or blocks the phosphorylation of HSP90 and/or the 50 kD CTL protein and thereby select the agent the blocks the suppression of CTL activity against HSV-infected target cells.

In another aspect, the present invention provides methods for modulating CTL activity. In one embodiment, the invention provides a method for blocking suppression of CTL activity against HSV-infected target cells comprising blocking the expression or functional activity of HSV U_{S}3. In another embodiment, a method is provided for suppressing CTL activity against a target antigen in a subject, the method including the following steps: (1) isolating a population of antigen presenting cells (APCs) presenting the target antigen on the cell surface; (2) introducing into the APCs an expression vector encoding an HSV U_{S}3 polypeptide and late HSV proteins, whereby the APCs express the HSV U_{S}3 polypeptide and late HSV proteins; and (3) administering to the subject the APCs expressing to the HSV U_{S}3 polypeptide and late HSV proteins, thereby suppressing CTL activity against the target antigen in the subject.

### DETAILED DESCRIPTION OF THE INVENTION

The present invention generally provides methods for screening an agent for activity in modulating T lymphocyte function as well as methods for methods of modulating T cell activity. In particular, the present invention provides methods for screening an agent for the ability to block the suppression of CD4⁺ and/or CTL activity against HSV-infected target cells. The invention relates to Applicants' surprising discovery that a cell infected with herpes simplex virus (HSV) are capable of inactivating a cytotoxic T lymphocyte (CTL) through a novel pathway that requires HSV U_{S}3 functional activity. Further, Applicants have discovered that heat shock protein 90 (HSP90) is tyrosine phosphorylated in HSV-inactivated T cells, causing it to disassociate from p56^{lck} and Raf, proteins that are necessary for proper T cell signaling. Still further, it has been demonstrated that TcR-stimulated phosphorylation of both proximal (i.e., TcR-ζ chain) and distal (i.e., ERK1,2) signaling intermediates were inhibited in HSV-inactivated T cells, as has calcium flux. The understanding of the molecular targets within lymphocytes that are manipulated by HSV to render the lymphocytes profoundly dysfunctional has provided targets for agents to inhibit or block the HSV-inactivation of the immune response to viral infection and other uses.

In one embodiment of the invention, the method of screening an agent for activity in modulating T lymphocyte activity comprises the following steps:
(1) contacting the agent with a cell infected with HSV, and/or contacting the agent with a T lymphocyte;
(2) contacting the HSV-infected cell with the T lymphocyte;
(3) contacting, in the absence of the agent, a second HSV-infected cell with a second CTL;
(4) determining for each of the first and second T lymphocytes the level of a physiological change associated with T lymphocyte function; and
(5) comparing the relative levels of the physiological change determined for each of the first and second T lymphocytes to determine whether the agent enhances or inhibits the physiological change, thereby determining whether the agent modulates T lymphocyte function.

In addition, it has also been observed that HSV-infected cells are capable of inactivating CD4⁺ T cells. Thus, in certain embodiments of the invention, CD4⁺ T cells are used rather than CD8⁺ CTLs in the methods as described herein.

In yet other embodiments of the screening methods, the cell contacted with the T lymphocyte is a recombinant cell that expresses an HSV U_{S}3 polypeptide and one or more other HSV proteins, a HSV-infected fibroblast, or other HSV-infected cell. Further, in certain embodiments, the T lymphocyte is also contacted with a second agent that activates the T cell receptor (TcR).

Prior to setting forth the invention in more detail, it may be helpful to a further understanding thereof to set forth definitions of certain terms as used hereinafter.

Definitions

Unless defined otherwise, all technical and scientific terms as used herein have the same meaning as commonly understood by one of ordinary skill in the art to which this invention belongs. Although any methods and materials similar to those described herein can be used in the practice or testing of the present invention, only exemplary methods and materials are described. For the purposes of the present invention, the following terms are defined below.

The term “biomolecule” as used herein refers to a molecule that is or can be produced by a living system as well as structures derived from such molecules. Biomolecules include, for example, proteins, glycoproteins, carbohydrates, lipids, [symbol] glycolipids, fatty acids, steroids, purines, pyrimidines, and derivatives, analogs, and/or combinations thereof.

The terms “agent” and “compound” as used herein are synonymous and are used to refer to a molecule, or a mixture of molecules, that are potentially capable of a physical interaction with a biomolecule through non-covalent interactions such as, e.g., through hydrogen bonds, ionic bonds, van der Waals attractions, or hydrophobic interactions. For example, compounds will most typically include molecules with functional groups necessary for structural interaction with, e.g., proteins, glycoproteins, or other biomolecules, particularly, for example, those groups involved in hydrogen bonding. Agents can include, for example, small organic molecules such as, e.g., aliphatic carbon or cyclical carbon (e.g., heterocyclic or carbocyclic structures and/or aromatic or polyaromatic structures). These structures can be substituted with one or more functional groups such as, for example, an amine, carbonyl, hydroxyl, or carboxyl group. In addition, these structures can include other substituents such as, for example, hydrocarbons (e.g., aliphatic, alicyclic, aromatic, and the like), nonhydrocarbon radicals (e.g., halo, alkoxy (e.g., methoxy, ethoxy), acetyl, carbonyl, mercapto, sulfoxy, nitro, amide, and the like), or hetero substituents (e.g., those containing non-carbon atoms such as, for example, sulfur, oxygen, or nitrogen). Compounds include those synthetically or biologically produced.

The term “target antigen” as used herein means an antigen that, when bound to an MHC class I or class II molecule, is specifically recognized by a T cell receptor (TcR) of a CTL or CD4⁺ T cell, respectively.

The term “T cell receptor-activating agent” (“TcR-activating agent”) as used herein means an agent that is capable of triggering activation of the T cell receptor of a CTL having functional components of the TcR signaling pathway.

The term “target cell” as used herein means a cell that is capable, upon contact with an activated CTL, of inducing the release of lytic granules from the activated CTL via activation of the T cell receptor. A target cell can, for example, express an MHC class I molecule bound to a target antigen, or can express Fcγ receptors, thereby inducing the release of lytic granules from, e.g., an anti-CD3 antibody-labeled CTL (see, e.g., Redirected Cell Lysis, Example 1, infra).

[symbol] in regulation of an immune response against a target antigen, particularly its function in killing cells having the target antigen (e.g., cells that have been infected by pathogens such as, for example, viruses encoding the target antigen), but also including other cell functions such as, for example, synthesis and release of cytokines (e.g., IFN-γ, TNF-α) involved in regulating the immune response.

The term “modulator of CTL function” as used herein refers to an agent that produces a physiological change associated with CTL function (e.g., an increase or decrease in a physiological response of the CTL) when the agent is contacted with the CTL and/or when the agent is contacted with another cell that interacts with the CTL (e.g., a cell expressing a HSV U_{S}3 polypeptide and late HSV proteins, including a HSV-infected cell).

The term “physiological change associated with CTL function” as used herein means any change in a cellular activity associated with or potentially associated with CTL function. The term includes, for example, intracellular physiological changes within the CTL, target cell, or other cell involved in an immunological response pathway mediated at least in part by a CTL. The term also includes physiological changes in the extracellular environment (e.g., presence of extracellular factors such as, for example, cytokines or chemokines). Examples of “physiological changes associated with CTL function” include, e.g., the release of cytokines (e.g., interferon-γ (IFN-γ), tumor necrosis factor-α (TNF-α), interleukin-2 (IL-2), interleukin-4 (IL-4), interleukin-10 (IL-10), interleukin-13 (IL-13)), exocytosis of lytic granules, expression of cell-surface molecules involved in immunological response pathways (e.g., CD3 or other components of the TcR, CD8, CD28, CTLA-4, CD40 ligand, 4-1BB, Fas, Fas ligand, CRAC (Ca²⁺ release activated Ca²⁺ channels)), or activation or inhibition of an intracellular signaling pathway (e.g., TcR signaling cascade, JAK-STAT pathways, or NFAT-associated pathways). In some preferred embodiments, the physiological change associated with CTL function is not a change in the expression of a cell-surface molecule. In other preferred embodiments, the physiological change associated with CTL function is a change in the expression of cytokines by the CTL, release of cytokines from the CTL, exocytosis of lytic granules from the CTL, or apoptosis of a target cell. In a specific embodiment of the present invention the physiological change associated with CTL function is a reduction or inhibition of phosphorylation of HSP90 and/or a 50 kD CTL protein in HSV-inactivated CTL.

As used herein, “CTL effector” refers to a molecule, complex of molecules, or subcellular structure that performs at least part of a CTL's function in the immune response against a target antigen (typically, e.g., function in cell killing), including, for example, cytokines (e.g., cytokines that enhance CTL-mediated cell killing such as, e.g. INF-γ and TNF-α) or perforin-containing lytic granules. Similarly, “CTL effector pathway” refers to a series of biochemical and/or cellular events involved in the performance of a CTL's function in the immune response against a target antigen.

The term “TcR signaling pathway” or “TcR signaling cascade” refers to a series of biochemical events initiated by TcR engagement (e.g., by antigen-MHC complex or anti-CD3 cross-linking) and that typically triggers (or contributes to triggering) one or more CTL effector pathways (by, for example, activating transcription of certain genes such as, e.g., genes encoding cytokines).

As used herein, the phrase “CTL having functional components of the TcR signaling pathway” means a CTL that exhibits sufficient activation of the TcR signaling cascade in response to cross-linking of the TcR (such as, e.g., cross-linking of the TcR using solid-phase anti-CD3 antibody), as determined using standard procedures to assess intracellular signaling events associated with TcR activation, including, e.g., Ca²⁺ release, phosphorylation of specific substrates, and specific protein-protein interactions.

As used herein, the phrase “CTL having functional components of TcR signaling and CTL effector pathways” means a CTL that exhibits sufficient activation of one or more CTL effector pathways (e.g., cytokine release or exocytosis of lytic granules) in response to cross-linking of the TcR (using, e.g., solid-phase anti-CD3 antibody) and activation one or more co-stimulatory pathways typically required for activation of the effector pathway (e.g., B7/CD28 pathway).

The term “apoptosis” refers to a regulated network of biochemical events that lead to a selective form of cell death and is characterized by readily observable morphological and biochemical phenomena, such as the fragmentation of the deoxyribo-nucleic acid (DNA), condensation of the chromatin, which may or may not be associated with endonuclease activity, chromosome migration, margination in cell nuclei, the formation of apoptotic bodies, mitochondrial swelling, widening of the mitochondrial cristae, opening of the mitochondrial permeability transition pores and/or dissipation of the mitochondrial proton gradient.

The term “polypeptide” refers to a polymer of amino acids and its equivalent and does not refer to a specific length of the product; thus, peptides, oligopeptides, and proteins are included within the definition of a polypeptide. A “fragment” refers to a portion of a polypeptide typically having at least 20, more typically at least 50, and still more typically at least 1000 or more contiguous amino acids of the polypeptide. For example, a U_{S}3 polypeptide fragment can be, e.g., at least 20, at least 50, at least 100, at least 200, or at least 300 or more contiguous amino acids of the native U_{S}3 polypeptide. A derivative is a polypeptide having conservative amino acid substitutions, as compared with another sequence. Derivatives further include, for example, glycosylations, acetylations, phosphorylations, and the like. Further included within the definition of “polypeptide” are, for example, polypeptides containing one or more analogs of an amino acid (e.g., unnatural amino acids, and the like), polypeptides with substituted linkages as well as other modifications known in the art, both naturally and non-naturally occurring. Ordinarily, such polypeptides will be at least about 50% identical to the native amino acid sequence, typically in excess of about 90%, and more typically at least about 95% identical.

The terms “polynucleotide” and “nucleic acid” refer to a polymer composed of a multiplicity of nucleotide units (ribonucleotide or deoxyribonucleotide or related structural variants) linked via phosphodiester bonds. A polynucleotide or nucleic acid can be of substantially any length, typically from about six (6) nucleotides to about 10⁹ nucleotides or larger. Polynucleotides and nucleic acids include RNA, cDNA, genomic DNA, synthetic forms, and mixed polymers, both sense and antisense strands, and can also be chemically or biochemically modified or can contain non-natural or derivatized nucleotide bases. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, and the like), charged linkages (e.g., phosphorothioates, phosphorodithioates, and the like), pendent moieties (e.g., polypeptides), intercalators (e.g., acridine, psoralen, and the like), chelators, alkylators, and modified linkages (e.g., alpha anomeric nucleic acids, and the like). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

The terms “amino acid” or “amino acid residue”, as used herein, refer to naturally occurring L amino acids or to D amino acids as described further below. The commonly used one- and three-letter abbreviations for amino acids are used herein (see, e.g., Alberts et al., Molecular Biology of the Cell (Garland Publishing, Inc., New York, 3rd ed. 1994).

In the context of a nucleic acid or polypeptide, the term “isolated” refers to a nucleic acid or polypeptide that has been at least partially removed from its natural cellular environment. For example, an isolated nucleic acid is typically at least partially purified from other cellular nucleic acids, polypeptides, and other constituents.

In the context of a cell, the term “isolated” refers to a cell that is at least partially removed from other constituents of its natural environment in vivo. For example, an isolated cell is at least partially purified from other constituents of the tissue from which the cell is derived (e.g., a hematopoietic cell such as, for example, a T cell, B cell, or dendritic cell from a blood sample is isolated where one or more other blood constituents (e.g., one or more other cell types) are removed).

The term “antigen presenting cell” (“APC”) as used herein refers to any cell capable of presenting a target antigen, bound to an MHC class I or class II molecule, on the cell surface. Preferably, the APC expresses or is capable of expressing MHC class I molecules. Further, in preferred embodiments, the APC expresses one or more other molecules typically required for CTL activation when the peptide-MHC complex is specifically recognized by the TcR of a CTL having functional components of the TcR signaling pathway (e.g., B7.1 or B7.2 (CD80 or CD86, respectively)).

The term “subject” as used herein means any mammalian patient to which the methods of modulating T lymphocyte activity (“immunomodulating methods”) as described herein may be administered. Subjects specifically intended for treatment using the immunomodulating methods include, for example, humans.

The term herpes simplex virus (HSV) as used herein refers to both human and non-human variants of HSV. Non-human variants include, for example, murine, rat, and bovine HSV.

The term “HSV U_{S}3 polypeptide” or “U_{S}3 polypeptide” as used herein refers to a polypeptide that is substantially identical or substantially similar to a polypeptide having the amino acid sequence set forth in SEQ ID NO:1 (Genbank Accession No. CAA52280; see Table 1) or SEQ ID NO:2 (Genbank Accession No. CAB06710; see Table 2) and having serine/threonine protein kinase activity. In certain embodiments of the invention, the U_{S}3 polypeptide is at least 70%, at least 80%, at least 90%, or at least 95% identical to a polypeptide having the amino acid sequence set forth in SEQ ID NO:1 or SEQ ID NO:2. In an exemplary embodiment, the U_{S}3 polypeptide is a polypeptide having the amino acid sequence set forth in SEQ ID NO:1 or SEQ ID NO:2. Kinase activity of the U_{S}3 polypeptide is determined using standard procedures known in the art, such as, e.g., in vitro kinase assays using isolated U_{S}3 polypeptide (e.g., U_{S}3 polypeptide immunoprecipitated with an anti-U_{S}3 antibody). In certain embodiments, the U_{S}3 polypeptide is capable of phosphorylating one or more of the HSV proteins, including, for example, ICP22, Us 1.5, U_{L}34, or U_{L}3.

TABLE 1

Amino Acid Sequence for HSV-1 Uₛ3 Protein Kinase
(SEQ ID NO:1)

macrkfcrvyggqgrrkeeavppetkpsrvfphgpfytpaedacldsppp
etpkpshttppseaerlchlqeilaqmygnqdypieddpsadaaddvded
apddvaypeeyaeelflpgdatgpligandhipppcgasppgirrrsrde
igatgftaeeldamdreaaraisrggkppstmaklvtgmgftihgaltpg
segcvfdsshpdypqrvivkagwytstshearllrrldhpailplldlhv
vsgvtclvlpkyqadlytylsrrlnplgrpqiaavsrqllsavdyihrqg
iihrdiktenifintpediclgdfgaacfvqgsrsspfpygiagtidtna
pevlagdpytttvdiwsaglvifetavhnaslfsaprgpkrgpcdsqitr
iirqaqvhvdefsphpesrltsryrsraagnnrppytrpawtryykmdid
veylvckaltfdgalrpsaaellclplfqqk

TABLE 2

Amino Acid Sequence for HSV-2 Uₛ3 Protein Kinase
(SEQ ID NO:2)

macrkfcgvyrrpdkrqeasvppetntapafpastfytpaedaylapgpp
etihpsrppspgeaarlcqlqeilaqmhsdedypivdaagaeeedeaddd
apddvaypedyaegrflsmvsaaplpgasghppvpgraappdvrtcdtgk
vgatgftpeeldtmdrealraisrgckppstlaklvtglgfaihgalipg
segcvfdsshpnyphrvivkagwyastshearllrrlnhpailplldlhv
vsgvtclvlpkyhedlytylskrpsplghlqitavsrqllsaidyvhckg
iihrdiktenifintpeniclgdfgaacfvrgcrsspfhygiagtidtna
pevlagdpytqvidiwsaglvifetavhtaslfsaprdperrpcdnqiar
iirqaqvhvdefpthaesrltahyrsraagnnrpawtrpawtryykihtd
veylickaltfdaalrpsaaellrlplfhpk

The term “HSV α proteins” or “HSV immediate early proteins” as used herein refers to products of a (i.e., immediate early) genes of HSV. HSV α genes are those genes expressed about 2 to about 4 hours postinfection at a multiplicity of infection (MOI) of 10 to 20 or expressed in the absence of other de novo synthesized viral proteins. HSV α proteins include ICP0, ICP4, ICP22, ICP27, ICP47 and U_{S}1.5. (See Fields Virology, Volume 2 (Knipe and Howley eds., Lippincott Williams and Wilkins, 4th ed. 2001) (herein “Knipe and Howley”).

The term “HSV β proteins” as used herein refers to products of β (i.e., early) genes of HSV. HSV β genes, which include β₁ and β₂ genes, are those HSV genes that require at least the presence of a functional ICP4 protein but not the onset of viral DNA synthesis. β₁ genes typically appear early after or substantially concurrently with the onset of α proteins and include, e.g., U_{L}29 (encoding ICP8), the gene encoding the single stranded-protein, and U_{L}39 (encoding ICP6, the large subunit of ribonucleotide reductase). β₂ genes are those typically exhibiting a substantial delay in expression after a protein synthesis and include, e.g., U_{L}23 (encoding viral thymidine kinase (TK)) and genes encoding components of the viral DNA polymerase (U_{L}30 and U_{L}42 proteins). (See Knipe and Howley, supra.)

The term “late HSV proteins” as used herein refers to protein products of γ₁ (“early-late” or “leaky-late”) and γ₂ (“true late”) genes, which are typically expressed at peak times after viral DNA synthesis has commenced and whose expression is enhanced by viral DNA synthesis. HSV γ₁ genes are typically expressed early in infection and only minimally affected by inhibitors of DNA synthesis. γ₁ genes include, for example, U_{L}19 (encoding ICP5), U_{L}27 (encoding gB), U_{S}6 (encoding gD), and the gene encoding ICP34.5. γ₂ genes are typically expressed at significant levels only after viral DNA synthesis and their expression is substantially inhibited in the presence of inhibitors of viral DNA synthesis. γ₂ genes include, for example, those encoding U_{L}36, U_{L}38, U_{L}41, U_{L}44 (gC), U_{S}11, U_{S}9, and U_{L}49.5. (See Knipe and Howley, supra.)

The term “functionally active,” in reference to U_{S}3 polypeptides or late HSV proteins, refers to those fragments, derivatives and analogs displaying one or more known functional activities associated with a full-length (wild-type) U_{S}3 polypeptide or HSV protein (e.g., serine/threonine protein kinase activity in the case of a U_{S}3 polypeptide).

The terms “identical” and “percent identity,” in the context of two or more nucleic acids or polypeptide sequences, refer to two or more sequences or subsequences that are the same or have a specified percentage of nucleotides or amino acid residues that are the same, when compared and aligned for maximum correspondence, as measured using one of the sequence comparison algorithms described infra or by visual inspection.

The phrase “substantially identical,” in the context of two nucleic acids or polypeptides, refers to two or more sequences or subsequences that have at least 60%, typically at least 70%, more typically at least 80%, and most typically at least 90% or at least 95% nucleotide or amino acid residue identity, when compared and aligned for maximum correspondence, as measured using one of the following sequence comparison algorithms, or by visual inspection.

“Similarity” or “percent similarity” in the context of two or polypeptide sequences, refer to two or more sequences or subsequences that are the same or have a specified percentage of amino acid residues or conservative substitutions thereof, that are the same, when compared and aligned for maximum correspondence, as measured using one of the sequence comparison algorithms described infra or by visual inspection. By way of example, a first amino acid sequence can be considered similar to a second amino acid sequence when the first amino acid sequence is at least 50%, at least 60%, at least 70%, at least 80%, at least 90%, or at least 95% identical, or conservatively substituted, to the second amino acid sequence when compared to an equal number of amino acids as the number contained in the first sequence, or when compared to an alignment of polypeptides that has been aligned by a computer similarity program known in the art, as discussed infra. In addition, a first amino acid sequence is considered substantially similar to a second amino acid sequence when the first amino acid sequence is at least 70%, preferably at least 80%, more preferably at least 85%, or most preferably at least 90% or at least 95% identical, or conservatively substituted, to the second amino acid sequence when compared to an equal number of amino acids as the number contained in the first sequence, or when compared to an alignment of polypeptides that has been aligned by a computer similarity program known in the art, as discussed infra. Thus, a polypeptide is substantially similar to a second polypeptide, for example, where the two peptides differ by one or more conservative substitutions.

The term “conservative substitution,” when describing a polypeptide, refers to a change in the amino acid composition of the polypeptide that does not substantially alter the polypeptide's activity. Thus, a “conservative substitution” of a particular amino acid sequence refers to substitution of those amino acids that are not critical for polypeptide activity or substitution of amino acids with other amino acids having similar properties (e.g., acidic, basic, positively or negatively charged, polar or non-polar) such that the substitution of even critical amino acids does not substantially alter activity. Conservative substitution tables providing functionally similar amino acids are well known in the art. For example, the following six groups each contain amino acids that are conservative substitutions for one another.
1) Alanine (Ala; A), Serine (Ser; S), Threonine (Thr; T);
2) Aspartic acid (Asp; D), Glutamic acid (Glu; E);
3) Asparagine (Asn; N), Glutamine (Gln; Q);
4) Arginine (Arg; R), Lysine (Lys; K);
5) Isoleucine (Ile; I), Leucine (Leu; L), Methionine (Met; M), Valine (Val; V); and
6) Phenylalanine (Phe; F), Tyrosine (Tyr; Y), Tryptophan (Trp; W).
   (See also Creighton, Proteins (W. H. Freeman and Company, 1984).) In addition, individual substitutions, deletions, or additions that alter, add or delete a single amino acid or a small percentage of amino acids in an encoded sequence are also “conservative substitutions.”

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm of Smith and Waterman (Adv. Appl. Math. 2:482, 1981), by the homology alignment algorithm of Needleman and Wunsch (J. Mol. Biol. 48:443, 1970), by the search for similarity method of Pearson and Lipman (Proc. Natl. Acad. Sci. USA 85:2444, 1988), by computerized implementations of these algorithms (e.g., GAP, BESTFIT, FASTA, and TFASTA in the Wisconsin Genetics Software Package, Genetics Computer Group, 575 Science Dr., Madison, Wis.), or by visual inspection. (See generally Ausubel et al., Current Protocols in Molecular Biology (John Wiley and Sons, New York, 4th ed. 1999)).

One example of a useful algorithm is PILEUP. PILEUP creates a multiple sequence alignment from a group of related sequences using progressive, pairwise alignments to show the percent sequence identity. It also plots a tree or dendogram showing the clustering relationships used to create the alignment. PILEUP uses a simplification of the progressive alignment method of Feng and Doolittle (J. Mol. Evol. 25:351, 1987). The method used is similar to the method described by Higgins and Sharp (Comput. Appl. Biosci. 5:151, 1989). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of an algorithm that is suitable for determining percent sequence identity and sequence similarity is the BLAST algorithm, which is described by Altschul et al. (J. Mol. Biol. 215:403, 1990). (See also Zhang et al., Nucleic Acid Res. 26:3986, 1998; Altschul et al., Nucleic Acid Res. 25:3389, 1997). Software for performing BLAST analyses is publicly available through the National Center for Biotechnology Information website. This algorithm involves first identifying high scoring sequence pairs (hsps) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer hsps containing them. The word hits are then extended in both directions along each sequence for as far as the cumulative alignment score can be increased. Extension of the word hits in each direction is halted when (a) the cumulative alignment score falls off by the quantity X from its maximum achieved value; (b) the cumulative score goes to zero or below, due to the accumulation of one or more negative-scoring residue alignments; or (c) the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff and Henikoff, Proc. Natl. Acad. Sci. USA 89:10915, 1992) alignments (B) of 50, expectation (E) of 10, M=5, N=−4, and a comparison of both strands.

In addition to calculating percent sequence identity, the BLAST algorithm also performs a statistical analysis of the similarity between two sequences (see, e.g., Karlin and [symbol] Altschul, Proc. Natl. Acad. Sci. USA 90:5873, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more typically less than about 0.01, and most typically less than about 0.001.

Screening Agents for Modulators of T Lymphocyte Function

In one aspect of the invention, an agent is screened for activity in modulating T lymphocyte function. In particular, embodiments the T lymphocytes are CD4⁺ lymphocytes or cytotoxic T lymphocytes (CTL). The method for screening an agent for T lymphocyte-modulating activity generally includes the following steps:
(1) contacting the agent with a cell expressing a HSV U_{S}3 polypeptide and one or more other HSV proteins (herein a “HSV U_{S}3-expressing cell” or “U_{S}3-expressing cell”), and/or contacting the agent with a T lymphocyte;
(2) contacting the HSV U_{S}3-expressing cell with the T lymphocyte;
(3) contacting a second HSV U_{S}3-expressing cell with a second T lymphocyte;
(4) determining for each of the first and second T lymphocytes the level of a physiological change associated with T lymphocyte function; and
(5) comparing the relative levels of the physiological change determined for each of the first and second T lymphocytes to determine whether the agent enhances or inhibits the physiological change, thereby determining whether the agent modulates T lymphocyte function.

Agents can be screened for activity in modulating T lymphocyte function either alone or in combination with other agents. Further, the methods can be used to screen any number of agents or combinations thereof for activity. Thus, in addition to testing a single agent, multiple agents such as, for example, agents from a compound library, can be screened for T-lymphocyte modulating activity using the methods described herein. Compounds [symbol] Compounds libraries can include, for example, a historical collection of compounds synthesized in the course of pharmaceutical research; libraries of compound derivatives prepared by rational design (see generally, e.g., Cho et al., Pac. Symp. Biocompat. 305, 1998; Sun et al., J. Comput. Aided Mol. Des. 12:597, 1998), such as, e.g., by combinatorial chemistry (see generally, e.g., DeWitt et al., Proc. Natl. Acad. Sci. USA 90:6909, 1993; International Patent Publication WO 94/0801; Burnbaum et al., Proc. Natl. Acad. Sci. USA 92:6027, 1995; Baldwin et al., J. Am. Chem. Soc. 117:5588, 1995; Nestler et al., J. Org. Chem. 59:4723, 1994; Borehardt et al., J. Am. Chem. Soc. 116:373, 1994; Ohlmeyer et al., Proc. Natl. Acad. Sci. USA 90:10922, 1993; and U.S. Pat. Nos. 5,958,792, 5,807,683, 6,006,617, and 6,077,954); natural products libraries, such as, e.g., those formed in the course of pharmaceutical research; peptide libraries; and the like.

The T lymphocytes used in the methods as described herein include T lymphocytes from, for example, mammals, birds, fish, and the like. In a preferred embodiment, cytotoxic T lymphocytes are used and the CTLs are mammalian CTLs, typically, e.g., human, murine, or rat, and most preferably human. In other preferred embodiments, the CTLs are mammalian CTL clonal cell lines. Mammalian CTL clones suitable for use in the methods described herein are known in the art and include, for example, the HLA-A *0201-restricted CD8⁺ CTL clone SKH-13, which recognizes the HA-8 minor histocompatability human antigen (mHA-8), and the HLA-A3-restricted CD8⁺ CTL clone KSN, which recognizes the peptide RVWDLPGVLK (SEQ ID NO:3).

Cells expressing HSV U_{S}3 polypeptide: Typically, cells expressing HSV U_{S}3 polypeptide are mammalian cells such as, e.g., human, rat, or murine cells. In certain embodiments, the U_{S}3-expressing cell is derived from a cell syngeneic to the T lymphocyte or CTL used in the assay; typically, these embodiments further ensure that receptor-ligand interactions are not minimized due to cross-species variation in receptor and/or ligand structure. In other embodiments, the U_{S}3-expressing cell is derived from a cell of a species different from that of the T lymphocyte or CTL. For example, in one embodiment of cross-species CTL inactivation using the methods herein, a human U_{S}3-expressing cell and a mouse CTL are used. Also, in certain embodiments, such as, for example, where the CTL is removed from the U_{S}3-expressing cell following contact (e.g., prior to contacting the CTL with a target cell or detecting the physiological change associated with CTL function), the U_{S}3 expressing cell is an adherent cell (e.g., a fibroblast, epithelial cell, or hepatocyte). [symbol] thereby facilitating the removal of the CTL from the HSV U_{S}3-expressing cell by, e.g., gentle aspiration of the CTL from the adherent cell.

In one embodiment, the cell expressing the HSV U_{S}3 polypeptide is a cell infected with a herpes simplex virus such as, for example, HSV-1 or HSV-2. The cell infected with HSV is typically a mammalian cell such as, e.g., a mammalian fibroblast, epithelial cell, or hepatocyte. In particular embodiments of the methods of the invention disclosed herein the infected cells are fibroblasts. Methods for infecting cells with HSV are known in the art. In certain embodiments, cells are infected with HSV at, e.g., an MOI (multiplicity of infection) of about 0.2, about 0.5, about 1, about 2, about 5, or about 10. For example, in one embodiment, adherent mammalian cells such as, e.g., fibroblasts or epithelial cells are grown to confluency (e.g., in a 96-well, flat-bottom plate) and infected with HSV at an MOI of 10 for 6 hours in cell culture medium (e.g., DMEM with 2% FCS), followed by one or more washes with PBS.

In other embodiments, the cell expressing the HSV U_{S}3 polypeptide is a recombinant cell comprising one or more expression vectors encoding the U_{S}3 polypeptide. Methods for nucleic acid manipulation, vector construction, and production of recombinant cells are generally known in the art and can be used to produce a recombinant cell expressing a HSV U_{S}3 polypeptide suitable for use in the methods described herein. (See generally, e.g., Sambrook and Russell, Molecular Cloning: A Laboratory Manual (Cold Spring Harbor Laboratory Press, Cold Spring Harbor, N.Y., 3rd ed. 2001); Sambrook et al., Molecular Cloning: A Laboratory Manual (Cold Spring Harbor Laboratory Press, Cold Spring Harbor, N.Y., 2nd ed. 1989); Ausubel et al., Short Protocols in Molecular Biology (Wiley and Sons, 4th ed. 1999)). Specific embodiments for, e.g., the isolation of a nucleic acid encoding a U_{S}3 polypeptide, insertion into cloning and expression vectors, and transfer of the expression vector into a cell are presented herein as examples and not by way of limitation.

Typically, the nucleic acid source for cloning of a U_{S}3 nucleic acid is HSV genomic or cDNA such as, for example, genomic or cDNA from HSV-1 or HSV-2 comprising the U_{S}3 gene. The DNA can be obtained by standard procedures known in the art from cloned DNA (e.g., a DNA “library”), by chemical synthesis, by cDNA cloning, or by the cloning of genomic DNA, or fragments thereof, purified from, e.g., HSV or an HSV-infected cell. (See, e.g., Sambrook and Russell, supra; Sambrook et al., supra; DNA Cloning: A Practical genomic DNA can contain regulatory and intron DNA regions in addition to coding regions; clones derived from cDNA will typically contain only exon sequences. Whatever the source, the nucleic acids can be molecularly cloned into a suitable vector for propagation of those nucleic acids.

For example, in one embodiment, polymerase chain reaction (PCR) can be used to amplify the desired U_{S}3 sequence from genomic DNA or cDNA. Oligonucleotide primers representing known U_{S}3 sequences can be used as primers in PCR. The synthetic oligonucleotides can be utilized as primers to amplify particular oligonucleotides within the U_{S}3 gene by PCR sequences from a source (RNA or DNA). PCR can be carried out, for example, by use of a Perkin-Elmer Cetus thermal cycler and Taq polymerase (GeneAmp®). The DNA being amplified can include mRNA or cDNA or genomic DNA derived from, e.g., HSV or an HSV-infected eukaryotic cell. Degenerate primers can also be synthesized for use in the PCR reactions.

Alternatives to isolating the U_{S}3 DNA include, but are not limited to, chemically synthesizing the gene sequence itself from a known sequence or making cDNA to the mRNA that encodes the U_{S}3 polypeptide. For example, RNA for cDNA cloning of a U_{S}3 gene can be isolated from cells that express a U_{S}3 polypeptide (e.g., eukaryotic cells infected with HSV).

The isolated U_{S}3 nucleic acids can then be inserted into an appropriate cloning vector. A large number of vector-host systems known in the art can be used. Possible vectors include, but are not limited to, plasmids, or modified viruses. The vector system is selected to be compatible with the host cell. Such vectors include, but are not limited to, bacteriophages such as lambda derivatives, yeast integrative and centromeric vectors, 21 plasmid, and derivatives thereof, or plasmids such as, e.g., pBR322, pUC, pcDNA3.1 or pRSET (Invitrogen) plasmid derivatives or the Bluescript vector (Stratagene). The insertion of the U_{S}3 nucleic acids into a cloning vector can be accomplished by known methods. (See, e.g., Sambrook and Russell, supra; Sambrook et al., supra; Ausubel et al., supra).

The nucleotide sequence coding for a U_{S}3 polypeptide, or a functionally active derivative, analog, or fragment thereof, can be inserted into an appropriate expression vector (i.e., a vector which contains the necessary elements for the transcription and translation of the inserted polypeptide-coding sequence). The necessary transcriptional and translational signals can also be supplied by the native U_{S}3 gene and/or its flanking regions. A variety of host-vector systems can be utilized to express the polypeptide-coding sequence. These include, but are not limited to, mammalian cell systems infected with virus (e.g., vaccinia virus, adenovirus, and the like), insect cell systems infected with virus (e.g., baculovirus), microorganisms such as yeast containing yeast vectors, or bacteria transformed with bacteriophage DNA, plasmid DNA, or cosmid DNA. The expression elements of vectors vary in their strengths and specificities. Depending on the host-vector system utilized, any one of a number of suitable transcription and translation elements can be used. In specific embodiments, a nucleic acid encoding the U_{S}3 polypeptide having the amino acid sequence set forth in SEQ ID NO:1 or SEQ ID NO:2, or a functionally active portion thereof, is expressed, in a mammalian cell.

Functional activity of the expressed polypeptide can be confirmed using, e.g., standard procedures known in the art. For example, the polypeptide can be tested in kinase assays such as, for example, in vitro kinase assays with the isolated polypeptide and a suitable substrate. Several suitable substrates for serine/threonine phosphorylation by a U_{S}3 polypeptide are known and include, for example, the immediate early regulatory proteins ICP22, U_{S}1.5, U_{L}34, and/or U_{L}3.

Expression of nucleic acid sequences encoding a U_{S}3 polypeptide, or a functionally active derivative, analog, or fragment thereof, can be regulated by a second nucleic acid sequence so that the U_{S}3 polypeptide or a functionally active derivative, analog, or fragment thereof is expressed in a host cell transformed with the recombinant DNA molecule. For example, expression of a U_{S}3 polypeptide can be controlled by any promoter/enhancer element known in the art. Promoters that can be used to control U_{S}3 gene expression include, but are not limited to, the SV40 early promoter region (Benoist and Chambon, Nature 290:304, 1981); the promoter contained in the 3′ long terminal repeat of Rous sarcoma virus (Yamamoto et al., Cell 22:787, 1980); the herpes thymidine kinase promoter (Wagner et al., Proc. Natl. Acad. Sci. USA 78:1441, 1981); the regulatory sequences of the metallothionein gene (Brinster et al., Nature 296:39, 1982); and the like.

Any of the methods previously described for the insertion of DNA fragments into a vector can be used to construct expression vectors containing a chimeric gene consisting of appropriate transcriptional/translational control signals and the polypeptide coding sequences. These methods include in vitro recombinant DNA and synthetic techniques and in vivo recombinants (genetic recombination). Expression vectors containing U_{S}3 nucleic acid inserts can be identified by general approaches well known to the skilled artisan, including, for example, (a) nucleic acid hybridization, (b) the presence or absence of “marker” gene function, and (c) expression of inserted sequences.

In a specific embodiment, a vector is used that comprises a promoter operably linked to a U_{S}3-encoding nucleic acid, one or more origins of replication, and, optionally, one or more selectable markers (e.g., an antibiotic resistance gene).

In addition, a host cell strain can be chosen that modulates the expression of the inserted U_{S}3 sequences, or modifies or processes the gene product in the specific fashion desired. Expression from certain promoters can be elevated in the presence of certain inducers; thus, expression of the genetically engineered U_{S}3 polypeptide can be controlled. Furthermore, different host cells having characteristic and specific mechanisms for the translational and post-translational processing and modification (e.g., glycosylation, phosphorylation) of polypeptides can be used. Appropriate cell lines or host systems can be chosen to ensure the desired modification and processing of the U_{S}3 protein expressed.

The U_{S}3-expressing cell also expresses one or more other HSV proteins. For example, the U_{S}3-expressing cell can also express one or more a regulatory proteins (e.g., ICP22, U_{S}1.5, U_{L}34, and/or U_{L}3), β proteins (e.g., ICP8, the single stranded-protein, ICP6, viral TK, U_{L}30, and/or U_{L}42 proteins), and/or late HSV proteins (γ1 and/or γ2 proteins) (e.g., ICP5, gB, gD, ICP34.5, U_{L}36, U_{L}38, U_{L}41, U_{L}44 (gC), U_{S}11, U_{S}9, and/or U_{L}49.5). In certain embodiments, the U_{S}3-expressing cell also expresses all HSV immediate early regulatory proteins, all β proteins, all γ1 proteins, and/or all γ2 proteins that are expressed over the full course of HSV infection of a eukaryotic cell. For example, in specific embodiments, the U_{S}3-expressing cell expresses, alternatively, all HSV proteins, all late HSV proteins (γ1 and γ2), or all β and γ₁ proteins expressed during the full course of HSV infection of a eukaryotic cell. In an exemplary embodiment, the cell expressing other HSV proteins with the U_{S}3 polypeptide is a cell infected with HSV (e.g., HSV-1 or HSV-2). In still other embodiments, the cell is a recombinant cell comprising one or more expression vectors encoding the U_{S}3 polypeptide and other HSV proteins. As set forth supra with respect to expression of recombinant U_{S}3, methods for nucleic acid manipulation, vector construction, and recombinant cell production are generally known in the art and can thus be used to produce a recombinant cell expressing a U_{S}3 polypeptide with other HSV proteins. (See generally, e.g., Sambrook and Russell, supra.; Sambrook et al., supra; Ausubel et al., supra.) For example, [symbol] the specific embodiments described above for the isolation of a polypeptide-encoding nucleic acid, insertion into cloning and expression vectors, and transfer of the expression vector into a cell can be used to produce, e.g., a recombinant cell expressing, in addition to a U_{S}3 polypeptide, all γ₁ HSV proteins and/or one or more immediate early regulatory proteins (e.g., ICP22, U_{S}1.5, U_{L}34, and/or U_{L}3).

T cell receptor-activating agents: In certain embodiments, the CTLs that have been contacted with a U_{S}3-expressing cell are also contacted with an agent that activates the T cell receptor (TcR-activating agent). TcR activation typically induces a variety of physiological changes associated with CTL function, including, for example, cytokine synthesis (e.g., IFN-γ and TNF-α) and exocytosis of lytic granules. TcR-activating agents include any agent capable, with respect to a CTL having functional components of the TcR and its downstream signaling effectors, of triggering the TcR complex and TcR signaling cascade. TcR-activating agents suitable for use in the methods described herein can include, for example, a superantigen such as, e.g., a staphylococcal enterotoxin (e.g., staphylococcal enterotoxin B (SEB)) or an antibody capable of cross-linking the TcR (e.g., an anti-CD3 antibody). In certain embodiments where the TcR-activating agent is an anti-CD3 antibody, the anti-CD3 antibody can be, e.g., immobilized on a solid support (such as, for example, on a 96-well flat-bottom tissue culture plate) (herein a “solid phase antibody”) or free in solution and subsequently cross-linked with, e.g., a secondary anti-Ig antibody following binding of the anti-CD3 antibody to the TcR. In other embodiments where the TcR-activating agent is an anti-CD3 antibody, the anti-CD3-labeled CTL is contacted with, e.g., a cell expressing on its surface Fcγ receptors (e.g., a FcγR⁺ P815 target cell), thereby triggering the TcR upon binding of the Fcγ receptors to the Fc region of the anti-CD3 antibody.

In other embodiments, the TcR-activating agent is a target antigen bound to an MHC class I molecule, where the target antigen is specifically recognized by the CTL used in the assay. Typically, the target antigen-MHC class I complex is presented on the surface of a target cell, and the CTL is contacted with the MHC-bound target antigen by contacting the target antigen-specific CTL with the target cell. Cells suitable for use as target cells in the methods described herein are known in the art and include, for example, EBV-transformed B cell lines such as, e.g., CEPH-8240 (positive for the minor histocompatability human antigen 8 (mHA-8) and which can be used, e.g., with the HLA-A *0201-restricted CD8⁺ CTL clone [symbol] can be used, e.g., with the HLA-A3-restricted CD8⁺ CTL clone KSN). In addition to target cells available in the art, target cells can also be prepared using known methods such as, for example, loading of an isolated target antigen peptide (e.g., synthesized by, for example, standard chemical methods known in the art) onto cells expressing MHC class I molecules.

Screening an agent for T lymphocyte-modulating activity: As set forth supra, to screen an agent for T lymphocyte-modulating activity, the agent is contacted with a cell expressing a U_{S}3 polypeptide, a T lymphocyte, or both a U_{S}3-expressing cell and a T lymphocyte. This step is typically performed, for example, prior to contacting the U_{S}3-expressing cell and the T lymphocyte; in these embodiments, the presence of the agent can, e.g., be maintained during part of or throughout the entire incubation of the U_{S}3-expressing cell and T lymphocyte. In certain alternative embodiments, the agent is added, for example, at a time that is substantially contemporaneous with (e.g., within 30 seconds, 1 minute, 2 minutes, or 5 minutes of) the contacting of the U_{S}3-expressing cell with the T lymphocyte. In certain embodiments of the present invention the T lymphocyte is a CTL or a CD4⁺ lymphocyte.

In certain embodiments, the T lymphocyte is removed from contact with the U_{S}3-expressing cell (by, for example, aspiration) after a desired time period and prior to detecting a physiological change or contacting the T lymphocyte with a TcR-activating agent. Alternatively, e.g., following an initial co-incubation of the T lymphocyte and U_{S}3-expressing cell for a desired time period, the cells are maintained in contact and/or are co-incubated together in culture during one or more of the screening method steps as described herein (e.g., contact with a TcR-activating agent or detection of a physiological change associated with CTL function). For example, the T lymphocyte and U_{S}3-expressing cell can be contacted, e.g., for about 1 hour, about 2 hours, about 3 hours, about 4 hours, about 5 hours, or about 6 hours prior to, e.g., removal of the T lymphocyte from the U_{S}3-expressing cell and/or detection of the physiological change associated with CTL function or, alternatively, prior to removal of the T lymphocyte from the U_{S}3-expressing cell and/or contacting the T lymphocyte with a TcR-activating agent (see supra).

In those embodiments in which a TcR-activating agent is used in the screening method for CTL activity, the TcR-activating agent is typically contacted with the CTL for a time period sufficient to trigger, in a CTL having functional components of the TcR signaling pathway, activation of the TcR complex and downstream components of the TcR signaling [symbol] least about 15 minutes or at least about 30 minutes). In addition, in certain embodiments, the TcR-activating agent is contacted with the CTL for a time period sufficient to trigger (e.g., at least about 15 minutes or at least about 30 minutes, and typically at least about 1 hour, at least about 2 hours, at least about 3 hours, at least about 4 hours, or at least about 5 hours), in a CTL having functional components of TcR signaling and effector pathways, activation of the TcR signaling complex, downstream components of the TcR signaling cascade, and one or more effector pathways of CTL function downstream of TcR activation (for example, expression of a cytokine (e.g., IFN-γ, TNF-α), expression of a cell surface receptor (e.g., CD-28, CTLA-4, 4-1BB), or release of lytic granules. In one embodiment, for example, the CTL and a target cell are co-incubated for a period of about 5 or about 6 hours.

Following the contacting of the CTL with the U_{S}3-expressing cell, or following the contacting of the CTL with a TcR-activating agent, the CTL is monitored for a physiological change associated with CTL function. In certain embodiments, where a target cell is used as a TcR-activating agent, the monitoring for a physiological change can include monitoring of the target cell for physiological change associated with CTL function. As set forth supra, physiological changes associated with CTL function include, for example, expression and/or release of cytokines (e.g., INF-γ, TNF-α), presence of lytic granule components in the CTL (e.g., granzymes or performs), exocytosis of lytic granules, changes in the phosphorylation state of a cellular protein (e.g., a decrease in the phosphorylation of heat shock protein 90 (HSP90) and/or a 50 kD CTL protein), or changes associated with apoptosis of a target cell.

Procedures for detecting physiological changes associated with CTL function are generally known in the art. For example, lysis of ⁵¹Cr-labeled target cells can be detected using a scintillation counter to measure counts per minute (cpm) in tissue culture supernatant removed from the cells. In addition, lytic granule content of CTLs can be determined by, e.g., staining the CTL with a labeled antibody specific for a component of CTL lytic granules (e.g., FITC-conjugated anti-granzyme A antibody using, for example, the Granzyme A reagent kit (clone CB9, BD Pharmingen, San Diego, Calif.)) and then detecting the label (using, e.g., flow cytometry in the case of a fluorescent label). Further, cytokine production can be detected by, for example, ELISA, Western blotting, flow cytometry, quantitative PCR, RNase protections assays, and the like. In one embodiment, for example, CTL, treated with, e.g., brefeldin A to prevent cytokine secretion, are fixed, permeabilized, and stained with labeled [symbol] FITC-conjugated anti-TNF-α clone 6401.1111) and then analyzed by flow cytometry. The CTL can also, for example, be labeled with a CTL-specific marker such as, e.g., a labeled anti-CD8 antibody (e.g., PerCP-conjugated anti-CD8 clone SK1). Also, phosphorylation of a cellular protein can be determined by, for example, immunoprecipitation of the protein followed by detection of the protein's phosphorylation state using, e.g. a phosphoprotein-specific antibody.

In addition to ⁵¹Cr-release, killing of a target cell can be monitored using any of various markers associated with apoptosis. For example, activation of components of apopotosis-specific pathways can be detected using, e.g., methods known in the art. For example, in one embodiment, activated caspase 3 is detected by staining with an activated caspase 3-specific antibody (e.g., using the Active Caspase-3 FITC MAb Apoptosis Kit I (BD PharMingen, San Diego, Calif.)). Other apoptosis-related physiological changes that can be measured in a target cell include, for example, cell shrinkage, chromosome condensation and migration, mitochondria swelling, disruption of mitochondrial transmembrane potential (measured, e.g., by staining with MitoTracker® Red CMXRos (Molecular Probes, Inc., Eugene, Oreg.), and/or cell death (measured, e.g., by a dye exclusion assay using, for example, propidium iodide (PI) or trypan blue).

As a control, a CTL is contacted with a U_{S}3-expressing cell in the absence of any contact with the agent, followed by contact of the CTL with a TcR-activating agent (optionally) and detection of a physiological change associated with CTL function as described supra.

To determine whether the agent has activity in modulating CTL function, the level of the physiological change measured for each of the agent-treated CTL (or CTL contacted with an agent-treated U_{S}3-expressing cell) and the control CTL are compared to determine whether a decrease or increase in the physiological change is associated with the presence of the agent. For example, the agent is generally identified as a stimulator of CTL function where the CTL that has been contacted with the agent (and/or contacted with a U_{S}3-expressing cell that has been contacted with the agent) exhibits a greater level of the physiological change as compared to the control CTL (e.g., increased cytokine production or release, decreased lytic granule content, increased activation of apoptotic pathways in the target cell, increased target cell lysis, and the like). Of particular interest are those agents that prevent or inhibit the phosphorylation of HSP90 and/or a 50 kD CTL protein in HSV-inactivated [symbol] agent. Also of particular interest are those agents that induce the phosphorylation of HSP90 and/or a 50 kD T lymphocyte protein irrespective of contact with a U_{S}3 expressing cell. These agents would be expected to suppress T cell function.

Methods of Modulating T Lymphocyte Function

Blocking suppression of T lymphocyte activity: In another aspect, the present invention provides a method for blocking the suppression of CTL activity against HSV-infected target cells (i.e., a method for blocking HSV-mediated inactivation of CTL). The method for blocking HSV-mediated inactivation generally comprises blocking the expression or functional activity of HSV U_{S}3 polypeptide. For example, in one embodiment, the expression of HSV U_{S}3 polypeptide is blocked in the infected target cell using an U_{S}3 antisense nucleic acid. In other embodiments, the HSV U_{S}3 polypeptide is blocked using an agent identified as a stimulator of CTL function using one of the methods for screening an agent for CTL-modulating activity as described supra.

Suppressing CTL activity against a target antigen: In yet another aspect, the present invention provides methods for suppressing CTL activity against a target antigen in a subject, the method generally comprising the following steps:
(1) isolating a population of antigen presenting cells (APCs) presenting a target antigen on the cell surface;
(2) introducing into the APCs an expression vector encoding a U_{S}3 polypeptide and one or more other HSV proteins, such that the APCs express the U_{S}3 polypeptide and one or more other HSV proteins; and
(3) administering to the subject the APCs expressing the U_{S}3 polypeptide and one or more other HSV proteins to suppress CTL activity against the target antigen in the subject.

The isolated APCs express or can be induced to express MHC class I or class II molecules on the cell surface. In certain embodiments, the isolated APC population comprises, for example, a population of dendritic cells. The APCs can be isolated from any tissue in which they reside (e.g., blood) using, for example, known methods. Once the APC population is isolated, target antigens are loaded onto the MHC class I or class II molecules by exposing the cells to the target antigen in vitro (e.g., adding peptides comprising the target antigen to the culture medium).

In certain embodiments, the U_{S}3 polypeptide and one or more other HSV proteins are encoded by the same vector. In other embodiments, the U_{S}3 polypeptide and one or more other HSV proteins are encoded by two or more different vectors. For example, in addition to an expression vector encoding the U_{S}3 polypeptide, an expression vector encoding one or more other HSV proteins, as described supra, can be introduced into the isolated cells. For example, in certain embodiments, an expression vector encoding one or more α, β, or γ HSV proteins is introduced into the cells.

In a preferred embodiment, the subject is a mammal such as, e.g., mouse, rat, or human. Further, the isolated cells are preferably syngeneic to the subject. For example, in one embodiment, antigen presenting cells such as, e.g., dendritic cells are isolated from a subject and then administered to the same subject following introduction of a vector encoding the U_{S}3 polypeptide and, optionally, other HSV proteins.

The following examples are provided merely as illustrative of various aspects of the invention and shall not be construed to limit the invention in any way.

### EXAMPLE 1

As shown in the following example, HSV-infected fibroblasts transmitted a functionally inhibiting signal to CTL without infecting the CTL or inducing apoptosis in the CTL. This process is referred to herein as HSV-mediated inactivation of CTL. The inactivating signal markedly decreased the cytotoxic and cytokine effector functions of CTL, and the inactivated phenotype was sustainable after CTL were removed from HSV-infected cells and treated with IL-2. Although inactivated CTL could not be stimulated through the TcR, they were capable of secreting IFN-γ when treated with a phorbol ester plus ionomycin. Also, HSV-infected fibroblasts required the expression of a viral protein kinase, U_{S}3, to transmit the inactivating signal to CTL. Use of this HSV-mediated inactivation model in screening assays allows the identification of agents that specifically modulate CTL function.

Materials and Methods

Cell Lines and Viruses: The HLA-A*0201-restricted CD8⁺ CTL clone, SKH-13, recognizes the HA-8 minor histocompatibility human antigen (mHA-8). The HLA-A3-restricted CD8⁺ CTL clone, KSN, recognizes the peptide RVWDLPGVLK (SEQ ID NO:3). For target cells (positive and control), the EBV-transformed B cell lines (BLCLs), CEPH-8240 (mHA-8 positive), SDK (mHA-8 negative), KSN-LCL (peptide positive), and GAO (peptide negative), were used. (SKH-13 and its associated cell lines are described in, e.g., Brickner et al., J. Exp. Med. 193:195, 2001). Both CTL clones were stimulated using a 14 day schedule as previously described. (See Brickner et al., supra; Brodie et al., Nat. Med. 5:34, 1999.) BLCLs were grown in RPMI 1640 supplemented with 4 mM HEPES, 3 mM L-glutamine, 10% fetal calf serum (FCS), 50 U/ml penicillin, and 50 μg/ml streptomycin. Fibroblasts were obtained from human foreskin samples and maintained in DMEM, 10% FCS, 50 U/ml penicillin, and 50 μg/ml streptomycin. Human fibroblasts were used in assays from passage 5 to 12. Vero cells were obtained from the American Type Culture Collection (Manassas, Va.) and grown in the same medium as fibroblasts. Cells were screened regularly for mycoplasma.

The wild-type viruses HSV-1 strain F (see Ejercito et al., J. Gen. Virol. 2:357, 1968) and HSV-2 strain HG52 were used as indicated herein. The mutant viruses were deletion-rescue pairs derived from HSV-1 F. The U_{L}54 (ICP27) deletion and rescue viruses, vBSΔ27 and vBSΔ27R, have been described by McCarthy et al. (J. Virol. 63:18, 1989). The U_{S}12 deletion and rescue viruses, ICP47Δ and ICP47ΔR, have been described by York et al. (Cell 77:525, 1994). The U_{S}3 deletion and rescue viruses, R7041 and R7306, were provided by B. Roizman. (See Purves et al., J. Virol. 61:2896, 1987). The U_{S}5 deletion and rescue viruses, RAS116 and RAS137, have been described by Jerome et al. (J. Virol. 73:8950, 1999). Because the α4 (ICP4) rescue virus was not available, the α4 deletion virus d120 was compared to HSV-1 parent strain KOS 1.1. (DeLuca et al., J. Virol. 56:558, 1985). All viruses were grown on Vero cells and titered using standard plaque assays.

Cell-Mediated Cytotoxicity Assays: Fibroblasts were grown to confluency in 96-well, flat-bottom plates and infected with HSV at a multiplicity of infection (MOI) of 10 or mock-infected. In some assays, fibroblasts and CTL were pre-treated with acyclovir or cyclohexamide (CHX) for the times and concentrations indicated (acyclovir and CHX from Sigma, St. Louis, Mo.). After a 6 h infection of fibroblasts with HSV in DMEM with 2% FCS (medium), fibroblasts were washed with PBS. CTL were added in 100 μl medium/well and incubated for 4 h at 37° C. and 5% CO₂. These CTL are restricted to MHC molecules not expressed on the inactivating fibroblasts, and thus the CTL do not recognize the fibroblasts. HA-8⁺ and HA-8⁻ BLCL target cells (targets) were labeled with 100-200 μCi of Na₂ ⁵¹CrO₄ (Amersham Pharamcia Biotech, Piscataway, N.J.) for 2 to 4 h at 37° C., washed, and counted. If a 3-cell sandwich chromium release assay was performed, ⁵¹Cr-labeled targets in 100 μl/well were added to the HSV-infected fibroblasts and CTL at various effector to target [symbol] (E:T) ratios. Alternatively, HSV-inactivated CTL were gently aspirated from HSV-infected fibroblasts. Visual inspection confirmed that fibroblasts were not removed during aspiration. Removed CTL were counted and added to new wells containing ⁵¹Cr-labeled targets. CTL were incubated with ⁵¹Cr-labeled targets for 5 to 6 h at 37° C. 40 μL of supernatant/well was removed, added to a LumaPlate™ and counted in a scintillation counter (Packard, Meriden, Conn.; counts per minute=cpm). Spontaneous and maximal release were measured by adding medium or 5% oxtylphenyl-polyethylene glycol (IGEPAL®; Sigma, St. Louis, Mo.)) to ⁵¹Cr-labeled targets. The spontaneous release was always less than 20% of the maximal release. All wells were done in triplicate and percent specific ⁵¹Cr release (% SR) was calculated by the formula % SR=([mean experimental cpm−mean spontaneous cpm]/[mean maximal cpm−mean spontaneous cpm])×100. Percent inactivation=([% SR from control−% SR from inactivated]/% SR from control)×100, where the control group was CTL incubated on mock-infected fibroblasts and the inactivated group was CTL incubated on HSV-infected fibroblasts.

Redirected Cell Lysis: CTL were incubated with mock or HSV-infected fibroblasts and removed as described above. CTL were then incubated with a monoclonal antibody raised against CD3 (clone OKT3, Ortho-McNeil, Redwood City, Calif.) at 1 μg/ml for 15 m at 37° C. P815 cells over-expressing Fcγ receptor (see Maziarz et al., Int. J. Cancer 48:142, 1991) were labeled with 100-200 μCi of Na₂ ⁵¹CrO₄ for 2 h at 37° C., washed, counted and then added to OKT3 bound CTL at various E:T ratios for 6 h.

Flow Cytometric Analysis of Granzyme A and Cytokine Production: CTL were incubated with mock or HSV-infected fibroblasts and removed. Lytic granule content in CTL was determined by incubating CTL with BLCL at an E:T ratio of 1 to 5 for 5 h. Cells were then stained with peridinin-chlorophyll-protein complex-(PerCP)-conjugated anti-CD8 (clone SK1, BD PharMingen, San Diego, Calif.) and FITC-conjugated anti-granzyme A antibody using the Granzyme A reagent kit (clone CB9, BD PharMingen, San Diego, Calif.) and analyzed by flow cytometry.

To detect cytokine production, CTL were incubated with mock or HSV-infected fibroblasts as previously described, removed and added to unlabeled BLCL target cells at various E:T ratios for 6 h at 37° C. In some experiments, CTL were untreated, treated with 1 μg/ml staphylococcal enterotoxin B (SEB), or treated with 100 ng/ml phorbol 12-myristate [symbol] Sigma, St. Louis, Mo.). During the final 4 h of stimulation with BLCL or mitogens, 10 μg/ml brefeldin A (BFA, Sigma, St. Louis, Mo.) was added to prevent protein secretion and allow visualization of the retained cytokines. CTL were fixed, permeabilized and stained with PerCP-conjugated anti-CD8 (clone SK1), FITC-conjugated anti-IFN-γ (clone 25723.11), or FITC-conjugated anti-TNF-α (clone 6401.1111) following the manufacturer's suggested protocol (BD PharMingen, San Diego, Calif.) and analyzed by flow cytometry.

Flow Cytometric Analysis for T cell Signaling and Activation-Associated Cell Surface Antigens and Ligands: CTL were incubated with mock or HSV-infected fibroblasts, removed, and stained with FITC, PE, ECD, or PC5-conjugated antibodies. Antibodies from Beckman Coulter (Hialeh, Fla.) recognized: CD2 (clone T11), CD3 (UCHT1), CD5 (BL1a), CD7 (3A1E-12H7), CD8 (SFCi21Thy2D3 and B9.11), CD28 (CD28.2), CD38 (T16), CD45 (J.33), and CD56 (N901). Antibodies from Becton-Dickinson (San Jose, Calif.) recognized: CD5 (L17F12), CD25 (2A3), CD56 (MY31), CD57 (HNK-1), and HLA-DR (L243). Four-Color flow cytometry was performed on a Coulter XL instrument and data was compensated and analyzed using software developed in the University of Washington Hematopathology Laboratory by Dr. Brent L. Wood (see Kussick and Wood, Arch. Pathol. Lab. Med. 127:1140, 2003).

Levels of MHC class I-like molecules (MIC) were measured on mock and HSV-infected fibroblasts at 6 h and 10 h post-infection with a mAb specific for MICA and MICB (clone 6D4) and analyzed with flow cytometry as previously described (see Groh et al., Science 279:1737, 1998). A blocking antibody against CD94 (clone HP-3B1, Beckman Coulter, Hialeh, Fla.) was added to control and inactivated CTL at 5 μg per 1.5×10⁶ cells and incubated for 1 h prior to adding BLCL targets at an E:T of 2 in a cell mediated cytotoxicity assay (see Aramburu et al., J. Immunol. 144:3238, 1990).

Flow Cytometric Analysis of Apoptotic Markers: CTL were incubated with mock or HSV-infected fibroblasts, removed, and added to BLCL. To distinguish BLCL from CTL, BLCL were labeled with an integral membrane dye using the PKH26 Red Fluorescent Cell Linker Kit (Sigma, St. Louis, Mo.). CTL and PKH26-labeled BLCL were incubated at various E:T ratios for 5 h and activated caspase 3 was measured using the Active Caspase-3 FITC Mab Apoptosis Kit I (clone C92-605, BD PharMingen, San Diego, Calif.). Cells were then paraformaldehyde fixed and analyzed by flow cytometry (see Jerome et al., Nat. Med. 9:2, 2003).

[symbol]

Mitochondrial potential was assessed in untreated CTL, CTL treated with 1 μM staurosporine (Sigma, St. Louis, Mo.) for 4 h, CTL incubated with uninfected fibroblasts (control) for 4 h, and CTL incubated with HSV-infected fibroblasts (inactivated) for 4 h. CTL were then stained using MitoTracker® Red CMXRos as per the manufacturer's instructions (Molecular Probes, Inc., Eugene, Oreg.). Control and inactivated CTL were also stained with antibody against CD95/Fas (clone DX2, Dako, Carpinteria, Calif.) and with the DNA-binding dye 7-amino-actinomycin D (7-AAD, Sigma, St. Louis, Mo.) and analyzed by flow cytometry.

Quantitative Real-Time PCR Using TaqMan® Chemistry: CTL were incubated with uninfected or HSV-infected fibroblasts for 4 h, removed by aspiration, and incubated for 5 h at 37° C. In the acyclovir group, fibroblasts were treated with 50 μM acyclovir for 2 h prior to infecting with HSV for 6 h. Removed CTL were then incubated with CD8-conjugated Dynabeads® (Dynal A.S., Oslo, Norway) for 30 m rocking at 4° C. and washed as directed by the manufacturer. DNA was purified from uninfected fibroblasts, HSV-infected fibroblasts, and CD8 enriched CTL using DNeasy® Tissue Kits (Qiagen, Valencia, Calif.). DNA was quantified by spectrophotometry and used as template in TaqMan® real-time PCR reactions with primers and fluorogenic probes specific for the HSV glycoprotein B gene or the cellular beta-globin gene. Reactions were carried out using an ABI 7700 Sequencer (Applied Biosystems, Foster City, Calif.), and the sequences and concentrations of the primers and probes used were as previously described (Liu et al., J. Virol. 75:11178, 2001).

Results

Effect of Incubating CTL with Fibroblasts Infected with HSV-1 or HSV-2: To evaluate the possibility that HSV-infected cells might send an inactivating signal to CTL, a confluent fibroblast monolayer was mock-infected or infected with HSV-1 or HSV-2 for 6 h and washed. A CD8⁺ CTL clone that is restricted to MHC molecules not expressed on the fibroblasts was then added (i.e., there was no killing of mock or HSV-infected fibroblasts). After incubating the HSV-infected fibroblasts and CTL for 4 h, ⁵¹Cr-labeled LCL allogeneic target cells (HA-8 positive) were added, and the 3-cell sandwich was incubated for an additional 5 h. ⁵¹Cr release was measured in triplicate wells for each data point. At a multiplicity of infection (MOI) of 10 (i.e., 10 plaque forming units of HSV per fibroblast) and a wide range of effector to target (E/T) ratios, both HSV-1-infected and HSV-2-infected [symbol] targets. This phenomenon is referred to as HSV-mediated inactivation. When fibroblasts were infected for 6 h with HSV-2 at variable MOIs and CTL incubated for 4 h with the HSV-infected fibroblasts, HSV-mediated inactivation occurred at a MOI as low as 0.2, suggesting a very robust negative signaling event between HSV-infected fibroblasts and CTL. To determine the kinetics of inactivation, the HSV-2 infection and CTL incubation times were varied independently at an MOI of 10. At 2 h incubations of CTL with HSV-infected fibroblasts, the ability of HSV-infected fibroblasts to cause inactivation occurred as soon as 2 h after infection and plateaued by 6 h with greater than 90% reduction of ⁵¹Cr release. At 6 h post infection, inactivation was seen with as little as 1 h of CTL incubation with HSV-infected fibroblasts and was maximal by 3 h. ⁵¹Cr release from autogenic targets (HA-8 negative) was <1%.

The durability of the inactivated phenotype was also assessed. Fibroblasts were infected with HSV-2 or HSV-1 at a MOI of 10 for 6 h. CTL were incubated for 4 h with HSV-infected fibroblasts and removed by gentle aspiration. In addition to eliminating CTL contact with HSV-infected fibroblasts, CTL removal also decreased the potential for CTL infection during subsequent steps and eliminated the possibility that HSV-infected fibroblasts might directly influence or infect target cells. 80% to 90% of CTL were typically removed without removing fibroblasts as determined by microscopic inspection and counting. Removed CTL were incubated alone before adding ⁵¹Cr LCL target cells at an E:T ratio of 3 for 6 h, and they remained inactivated for at least 12 h. Treatment with IL-2 at 20 units/ml maintained the lytic ability of CTL added to mock-infected fibroblasts for up to 48 h, but CTL added to HSV-infected fibroblasts did not recover killing function even when treated with 20 units/ml IL-2.

In all subsequent procedures involving the HSV-mediated inactivation of CTL, fibroblasts were mock or HSV-infected for 6 h at a MOI of 10, and CTL were incubated for 4 h with the mock or HSV-infected fibroblasts. CTL from mock-infected fibroblasts (control) and HSV-infected fibroblasts (inactivated) were then removed from the fibroblasts before adding to target cells. When inactivated CTL were mixed with control CTL at ratios as high as 1:1, there was no suppression of lytic ability in the control CTL.

Although chromium release is a traditional method for determining the cytotoxic capacity of CTL, more biologically relevant methods have recently become available that examine apoptic markers, such as activated caspase [symbol] (2003), supra). Flow cytometry was used to assess activated caspase 3 levels in targets incubated with control or inactivated CTL. Target cells were first labeled with the integral membrane dye PKH26 to distinguish them from CTL. CTL were inactivated by incubating with HSV-2 infected fibroblasts, removed from infected fibroblasts, and then added to labeled target cells at an E:T ration of 1 before staining for activated caspase 3. In apoptotic cells, activated caspase 3 levels increase. In agreement with the chromium release data, targets incubated with inactivated CTL had substantially less activated caspase 3 relative to those incubated with control CTL.

Caspase activation in target cells occurs primarily through the exocytosis of lytic granules from activated CTL. These granules contain perforin and granzymes A and B (grA and grB). The SKH-13 CTL clone utilized this mechanism as well, since the addition of concanamycin, an agent that blocks granule exocytosis, prevented ⁵¹Cr release from target cells. grA levels in inactivated CTL were evaluated after incubation with targets. Unlabeled targets were added to CTL at an E:T ratio of 0.2. Cells were stained with antibody against CD8 and granzyme A and analyzed by flow cytometry gating for CD8 positive cells. Consistent with a previous assessment of cytotoxicity, there was markedly more grA remaining in the inactivated CTL relative to control CTL. Thus, HSV-inactivated CTL do not degranulate upon contact with target cells.

In addition to granule exocytosis, CTL control viral replication by synthesizing and secreting pro-inflammatory cytokines. CTL were inactivated as previously described with HSV-2 infected fibroblasts and removed before adding to target cells for 6 h at an E:T ratio of 0.2. To assess cytokine levels, CTL were treated with 10 μg/ml brefeldin A, an agent that prevents secretion of proteins, during the last 4 h of incubation with target cells. Cells were then stained with antibody against CD8, interferon gamma (IFN-γ), and tumor necrosis factor alpha (TNF-α) and analyzed by flow cytometry. Flow cytometric analysis revealed 90% less synthesis of IFN-γ and TNF-α in inactivated CTL relative to control CTL. Therefore, CTL were inhibited in at least 2 mechanistically distinct effector pathways in the HSV-mediated inactivation model.

Cell surface markers involved in T cell co-stimulation, accessory signaling, and activation were measured on control and inactivated CTL. There was no detectable difference by flow cytometry for the following molecules: CD2, CD3, CD5, CD7, CD8, CD19, CD25, CD28, CD38, CD45, CD56, CD57, and HLA-DR. An anti-CD94 blocking antibody added to CTL prior to incubating with HSV-infected fibroblasts did not alter inactivation, suggesting that the CD94-NKG2A molecules were also not involved. In addition, levels of the MHC class I-like molecules, MICA and MICB, were similar on mock and HSV-infected fibroblasts.

HSV-Mediated Inactivation Does Not Require Infection of CTL: One mechanism for the HSV-mediated inactivation involves a negative signaling event between infected fibroblasts and uninfected CTL. To determine if viral replication was necessary in HSV-infected fibroblasts and to decrease the possibility of cell-to-cell spread of virus to CTL, fibroblasts were treated with acyclovir prior to infection. Fibroblasts were untreated or treated with 50 μM or 500 μM acyclovir for 2 h prior to a 6 h infection with HSV-2. CTL were added to HSV-infected or mock-infected fibroblasts for 4 h and then removed by aspiration. Control and inactivated CTL were added to target cells at an E:T ratio of 3 for 6 h. HSV-infected fibroblasts retained the capacity to inactivate CTL even in the presence of 50-500 μM acyclovir. These results show that viral replication is not necessary for CTL inactivation by HSV-infected cells and that γ2 viral genes, which require viral DNA synthesis for their expression, are not required for inactivation in this model.

While acyclovir treatment substantially decreases the production of virus from infected cells, it did not rule out the possibility that HSV-inactivated CTL become infected in the HSV-mediated inactivation model. The most sensitive technique available to detect the presence of HSV is real-time, quantitative PCR. Primers and probes against a viral gene (gB) and a cellular gene (beta globin) were used to determine the number of HSV copies per cell in HSV-infected fibroblasts and in inactivated CTL. Fibroblasts were untreated or treated with 50 μM acyclovir prior to infection. CTL were then inactivated as before, removed, and enriched using CD8-coupled magnetic beads to decrease possible contamination with HSV-infected fibroblasts. Although fibroblasts treated with acyclovir and infected with HSV were capable of inactivating CTL, only 1 copy of HSV was detected per 6 CTL in this group, a value that approached the mock-infected background level of 1 copy of HSV per 29 fibroblasts (Table 3). In the group without acyclovir treatment, HSV-infected fibroblasts contained 4344 copies of HSV per cell. CTL incubated on HSV-infected fibroblasts contained 6.5 copies of HSV per cell (Table 3), suggesting that cell-to-cell spread of HSV from infected fibroblasts to CTL was a relatively rare event even in the absence of acyclovir. In summary, it is clear that HSV-mediated inactivation of CTL can occur in the absence of CTL infection.

TABLE 3
Quantitation of HSV levels in fibroblasts and
CTL using real-time PCR^{a}

| Cell type^{b} | # HSV Copies/cell^{c} |
|---|---|
| Mock-infected fibroblasts | 0.04 |
| CTL incubated on HSV-FB + acyclovir | 0.16 |
| HSV-infected fibroblasts (HSV-FB) | 4334 |
| CTL incubated on HSV-FB | 6.5 |

^{a}TaqMan ® chemistry used previously published primers and probes against HSV glycoprotein B (gB) gene and the cellular β-globin gene.
^{b}Fibroblasts, untreated or treated with 50 μM acyclovir, were infected for 6 h with HSV-2 or mock-infected. CTL were added for 4 h and removed, then incubated for 5 h alone. CTL were enriched using CD8-coupled magnetic beads.
^{c}DNA was extracted from cells and used as template. # HSV copies was determined by Cₜ values for gB at a given template dilution, and # cells was determined by Cₜ values for β-globin at the same template dilution.

Analysis of Apoptosis in HSV-Inactivated CTL: The same numbers of CTL were recovered by aspiration from HSV-infected fibroblasts as from uninfected fibroblasts, and trypan blue staining revealed no evidence of preferential loss of viability in CTL after contact with HSV-infected fibroblasts. Because this is a crude indicator of cell health, activated caspase 3 levels in CTL were measured by flow cytometry to determine if apoptosis played a role in HSV-mediated inactivation of CTL. CTL were inactivated as before and removed from HSV-2 infected fibroblasts before adding to PKH26 labeled target cells at an E:T ratio of 1 for 5 h. Cells were stained with antibody against activated caspase 3 and analyzed by flow cytometry. CTL were selected by gating for PKH26 negative events. The percentage of control and inactivated CTL staining positive for activated caspase 3 was virtually equal.

Another indicator of programmed cell death is the loss of mitochondrial membrane potential. CMXRos is a charge sensitive fluorochrome that binds to the inner mitochondrial membrane and decreases in fluorescence as membrane potential is lost. CTL were either (a) inactivated on HSV-2-infected fibroblasts as previously described or (b) incubated without fibroblasts, either alone or in the presence of 1 μM staurosporine (STS), for 4 h. All CTL were subsequently stained with CMXRos and analyzed by flow cytometry. HSV-inactivated CTL had a mitochondrial potential very similar to control CTL, whereas CTL treated with staurosporine, an agent that induces apoptosis through the mitochondrial pathway, showed a significant decrease in the CMXRos signal. Additionally, flow cytometry revealed no detectable difference in CD95/Fas levels or binding of 7-amino-actinomycin D in inactivated and control CTL. Taken together, these results show no evidence of increased programmed cell death in CTL inactivated by HSV-infected cells.

Stimulating HSV-Inactivated CTL through the TCR or with a Phorbol Ester Plus Ionomycin: TcR- and non-TcR-mediated stimulation of HSV-infected CTL were evaluated in three assays: (1) treatment with staphylococcal enterotoxin B (SEB), a super antigen that functions through TcR complex triggering; (2) a redirected lysis assay using anti-CD3 antibody; and (3) treatment with phorbol myristate acetate (PMA) plus ionomycin, a non-TcR-mediated method of stimulation. For each assay, CTL were inactivated and removed from HSV-infected fibroblasts (see supra) prior to treatment as described below. Taken together, the findings from these assays show that, although HSV-inactivated CTL could not be stimulated through the TcR, the HSV-inactivated CTL were viable and retained the capacity to functionally respond to other signaling pathways. For each assay, CTL were inactivated and removed as described supra.

1. SEB Treatment: Inactivated and control CTL were untreated or treated for 6 h with 1 μg/ml SEB. The final 4 h was in the presence of brefeldin A (BFA) at 10 μg/ml to prevent protein secretion. Cells were stained with antibody against CD8 and IFN-γ and analyzed by flow cytometry. Incubation of inactivated CTL with SEB led to an increase in IFN-γ synthesis in control but not inactivated CTL.

2. Redirected Lysis: The capability of inactivated CTLs to release lytic granules was evaluated in a redirected lysis assay against target cells expressing abundant Fcγ receptors (FcγR). Inactivated or control CTL were incubated with antibody against CD3 at 1 μg/ml for 15 m before adding to ⁵¹Cr labeled FcγR⁺ P815 target cells at various E:T ratios for 5 h. Control but not inactivated CTL were capable of lytic granule release via redirected lysis using anti-CD3 antibody labeled CTL and ⁵¹Cr labeled P815 cells that express abundant Fcγ receptors.

3. Treatment with PMA Plus Ionomycin: Inactivated or control CTL were treated with PMA at 100 ng/ml plus ionomycin at 1 μg/ml for 6 h (BFA at 10 μg/ml was added for the final 4 h). CTL were then stained with antibody against IFN-γ and analyzed by flow cytometry. Treatment of inactivated CTL with PMA plus ionomycin resulted in the production of IFN-γ levels comparable with control CTL.

Effect of UV Irradiation and Cyclohexamide (CHX) on HSV-Mediated Inactivation: In order to determine if the input virion was sufficient to inactivate CTL, the effect of HSV-UV-irradiation prior to fibroblast infection was examined. CTL were inactivated and removed as before and added to target cells at an E:T ratio of 3. HSV-2 was UV-irradiated on ice with 0.1 Joules in a STRATALINKER®. The control group consisted of CTL incubated with mock-infected fibroblasts. HSV and UV-HSV groups consisted of CTL incubated with fibroblasts infected with HSV or UV-irradiated HSV respectively. At energy levels as high as 0.1 joules, virus was still capable of cell entry; however, inactivation of CTL was virtually abolished. This indicated that de novo viral protein synthesis was required to inactivate CTL. To confirm these findings, CHX was used to independently inhibit translation in fibroblasts, CTL and target cells. Inactivation of CTL by HSV-infected fibroblasts was done as previously described except that each cell type was first treated with 10 μg/ml CHX. Fibroblasts were treated for 10 h prior to infection, CTL were treated for 5 h prior to incubating with fibroblasts, and targets were treated at the time of addition to CTL. While there was no effect on HSV-mediated inactivation when CTL or targets were exposed to CHX, treating the fibroblasts with CHX prior to infection abolished HSV-mediated inactivation. Therefore, protein synthesis is necessary in HSV-infected fibroblasts but not in CTL in order for inactivation to occur.

Analysis of HSV Deletion-Rescue Pairs in HSV-Mediated Inactivation of CTL: In HSV-infected cells, viral gene expression was divided temporally into 3 classes: immediate early genes (α), early genes (β) and late genes (γ). γ genes are further divided into γ₁ and γ₂, and acyclovir blocks expression of γ₂ but not γ₁ genes. The α genes, α4 and U_{L}54, act as transactivators and are required for the expression of most β and γ genes. To identify a HSV gene necessary to mediate inactivation, deletion-rescue pairs derived from the HSV-1 F parent strain were utilized. Fibroblasts were infected with deletion-rescue pairs deficient in either the α4, U_{L}54, U_{S}12, U_{S}3, or U_{S}5 gene. CTL were inactivated and removed from the HSV-infected fibroblasts as previously described and then added to labeled target cells at an E:T ratio of 3 for 5 h in a standard ⁵¹Cr release assay. Fibroblasts infected with α4 or U_{L}54 deletion viruses were incapable of inactivating CTL. Unlike α4 or U_{L}54, deletion of a non-transactivating α gene, U_{S}12, did not alter HSV-mediated inactivation. As predicted from the [symbol] acyclovir experiment where inactivation persisted, deletion of the γ gene U_{S}5 did not prevent inactivation. However, deletion of the β gene U_{S}3 markedly decreased inactivation and was comparable to deletions of α4 or U_{L}54. Thus, expression of U_{S}3 encoding the HSV protein kinase is required for inactivation of CTL.

Discussion

These results demonstrate that HSV-infected cells engage a signaling pathway in CD8⁺ CTL and inhibit their effector functions. CD4⁺ CTL have been found to be inactivated to a similar degree. CTL inactivated by HSV-infected cells lose the ability to release cytolytic granules and induce apoptosis in targets when triggered through the TCR. In addition, inactivated CTL secrete substantially less cytokines in the HSV-mediated inactivation model. Most CTL secrete the pro-inflammatory cytokine IFN-γ, which has a direct anti-viral effect and may serve to control viral replication by increasing expression of MHC class I on virally infected cells (see Liu et al., J. Virol. 75:11178, 2001; Kagi et al., Nature 369:31, 1994). CTL can also secrete TNF-α, which synergizes with IFN-γ to activate macrophages. Non-lytic mechanisms of CTL function are important in the pathogenesis of some diseases. For example, there are reports describing the critical role of IFN-γ in graft rejection. (See, e.g., Valujskikh et al., Nat. Immunol. 3:844, 2002). In addition, HIV specific CTL that become impaired in cytolytic function still retain the ability to secrete IFN-γ. (See Appay et al., J. Exp. Med. 192:63, 2000).

CTL inactivation occurs in the absence of CTL infection in HSV-mediated inactivation. Multiple approaches were used to show that inactivated CTL were not infected with HSV. For example, fibroblasts infected with HSV for 4 h inactivated CTL after a 2 h co-incubation, even after the CTL were removed and washed. Because there was no appreciable viral assembly or production after a 6 h HSV infection, HSV was not likely to spread from the fibroblasts to the CTL. Finally, inactivation occurred in the presence of acyclovir, and the number of viral copies detected by real-time PCR in inactivated CTL approached background levels.

HSV-mediated inactivation is apparently not a result of several known CTL silencing mechanisms. For example, results suggest that apoptosis was not responsible for CTL inactivation. Both control and inactivated CTL had similar levels of activated caspase 3 [symbol] did not bind the DNA binding dye 7-AAD. Also, HSV-mediated inactivation is not consistent with traditional anergy, as IL-2 did not rescue CTL function. Furthermore, the inactivated phenotype was not transferable, suggesting it was not a T cell suppressor-like phenomenon. However, agents that bypass the TcR, such as PMA and ionomycin, restored function in inactivated CTL suggesting that HSV-mediated inactivation is specific to the TcR.

CTL receive numerous signals in their progress through maturation, activation, proliferation, triggering, and persistence or elimination. Although the specificity of the activation and triggering processes is due to the TcR-CD3 complex, which binds a target peptide sequence in the context of classical MHC class Ia, TcR ligation alone was insufficient to activate CTL. Co-stimulatory signals such as CD28 and CD137 are necessary for CTL cytotoxicity, cytokine secretion, IL-2 production, and clonal expansion. (See, e.g., Chambers and Allison, Curr. Opin. Immunol. 9:396, 1997; Gimmi et al., Proc. Natl. Acad. Sci. USA 88:6575, 1991; Watts and DeBenedette, Curr. Opin. Immunol. 11:286, 1999). Co-stimulation is crucial since TcR ligation alone can lead to CTL anergy and apoptosis. (See, e.g., Harding et al., Nature 356:607, 1992). Therefore, a panel of T cell accessory, co-stimulatory, and activation markers were tested, including CD2, CD3, CD8, CD19, CD25, CD28, CD38, and CD45. No difference was found in the cell surface phenotype between control and inactivated CTL in HSV-mediated inactivation.

Recent reports have described growing families of related receptors on CTL that have opposing functions and facilitate the equilibrium between initiation and termination of a CTL response. These reports suggest that a complex equilibrium exists between CTL and cells they encounter at various points in the immune response. For example, the NK cell receptors (NKRs), such as CD94-NKG2, and the killer cell Ig-like receptors (KIRs) can initiate both positive and negative regulatory pathways by binding various MHC class I and MHC class I-like molecules (MIC) on APCs and target cells. (See Lanier, Curr. Opin. Immunol. 13:326, 2001; Ugolini and Vivier, Nat. Immunol. 2:198, 2001). One potential mechanism for HSV-mediated inactivation of CTL is that HSV-infected fibroblasts are up- or down-regulating a ligand for an inactivating or activating CTL receptor. However, no difference in MICA or MICB levels was detected on mock-infected and HSV-infected fibroblasts. Furthermore, CD94 blocking antibodies had no effect on HSV-mediated inactivation of CTL. There is evidence in the literature for altered expression of CTL regulatory receptors and ligands in stressed cells. For instance, elevated levels of MICA and MICB have been reported in CMV infected cells (see Groh et al., Nat. Immunol. 2:255, [symbol] 2001), while the inhibitory CD94-NKG2A receptor on CTL has been shown to be up-regulated in polyoma virus induced oncogenesis (see Moser et al., Nat. Immunol. 3:189, 2002). In addition, ligation of the CTL co-stimulatory molecule CD137, a TNF-receptor family member, can stimulate a potent anti-tumor response. (See Wilcox et al., J. Clin. Invest. 109:651, 2002; Melero et al., Nat. Med. 3:682, 1997).

Since UV irradiation of HSV or treatment of fibroblasts with cyclohexamide abolishes HSV-mediated CTL inactivation, new viral protein synthesis and possibly cellular protein synthesis is necessary for HSV-infected cells to transmit an inactivating signal. Additionally, acyclovir treated, HSV-infected fibroblasts maintain the ability to inactivate CTL. Thus, viral replication and 72 late gene expression, which requires viral DNA synthesis, are not essential. Deletion-rescue pairs further reveal which viral genes HSV-infected fibroblasts are required to inactivate CTL. The α transactivating genes control expression of numerous β and γ HSV genes. Deletions of the transactivating genes α4 or U_{L}54, encoding the proteins ICP4 and ICP27, abolished HSV-mediated inactivation, while deletion of a non-transactivating gene U_{S}12, encoding ICP47, did not alter inactivation. From these results, it is clear that one or more downstream viral genes (β or γ₁) are required to transmit the inactivating signal from HSV-infected fibroblasts to CTL. Deletion-rescue constructs corresponding to two of these genes, U_{S}3 and U_{S}5, were tested for their effect on HSV-mediated inactivation of CTL. As predicted from experiments using acyclovir, in which γ gene expression should have been minimal but inactivation still occurred, deletion of the γ gene U_{S}5 encoding gJ did not affect inactivation. Alternatively, the dramatic reduction of inactivation that resulted from the deletion of U_{S}3 is similar to the response seen with the deletion of α4 or U_{L}54. The U_{S}3 deletion virus grew as robustly as the rescue and parent virus, yet deletion of this single gene virtually eliminated HSV-mediated inactivation of CTL. This result, in conjunction with the ability of MOIs as low as 0.2 to induce inactivation, suggest that this phenomenon is not just a product of non-specific viral infection. Additionally, the U_{S}3 deletion-rescue pair provides a tool to systematically screen the altered membrane of HSV-infected cells to identify viral or cellular gene products responsible for transmitting the negative signal to CTL.

U_{S}3 is known to phosphorylate several proteins and affect their expression and function. Known viral substrates for U_{S}3 include the immediate early regulatory proteins ICP22 and U_{S}1.5, as well as U_{L}34, and U_{L}5.(See Ogle and Roizman, J. Virol. 13:4305, 1999; Poon et al., J. Virol. 74:11210, 2000; Markovitz et al., J. Virol. 73:8010, 1999). U_{S}3 has also been shown to inhibit apoptosis in HSV infected cells by modifying cellular proteins. (See Jerome et al., J. Virol. 73:8950, 1999; Murata et al., Microbes Infect. 4:707, 2002). U_{S}3 was reported to post-translationally modify BAD, a proapoptotic member of the Bcl-2 family, and prevent it from activating programmed cell death. (See Munger and Roizman, Proc. Natl. Acad. Sci. USA 98:10410, 2001). U_{S}3 may also play a role in the ability of HSV to cause productive infection in living organisms. Evidence for this can be found in an in vivo model of murine HSV-2 genital herpes, where there was more rapid viral clearance in mice infected with a U_{S}3-deficient mutant relative to a U_{S}2 mutant or wild-type virus. (See Inagaki et al., Vaccine 20:98, 2001). In the present example, the U_{S}3-deficient mutant produced an immune response that had larger number of CD8⁺ T cells and increased levels of secreted IFN-γ.

In HSV-mediated inactivation of CTL, the magnitude of CTL cytolytic impairment consistently exceeded 90%. The magnitude of impairment of CTL cytokine production was similar. Inhibition of two separate pathways (lytic granule release and cytokine synthesis) and the ability of PMA plus ionomycin to overcome inactivation indicate a more proximal or upstream signaling deficiency. Also, while activated CTL undergo apoptosis through Fas-Fas ligand engagement, and while HSV-infected CTL are more prone to apoptosis, CTL inactivated by HSV-mediated inactivation were neither apoptotic nor infected with HSV.

Further, the early gene U_{S}3 is required in HSV-mediated CTL inactivation. U_{S}3 encodes a protein kinase that can modify viral and cellular proteins, but it is not required for infectivity. While it is possible that the CTL inactivating signal from HSV-infected cells is an important feature in the successful ability of HSV to infect, replicate, and persist in vivo, this model also supports the use of HSV and U_{S}3 as tools to probe the molecular nature of a critical cellular signaling event and to screen for agents that specifically modulate CTL function. Such agents would be useful for stimulating dysfunctional CTL that exist in persistent viral infections and tumor states or, alternatively, for promoting inactivation of CTL in GVHD and autoimmune states in both human and non-human subjects.

### EXAMPLE 2

The following example demonstrates that heat shock protein 90 is tyrosine phosphorylated in Hepes simplex virus-inactivated T cells, causing it to disassociate from p56^{lck} and Raf, proteins that are necessary for proper T cell signaling. Consequently, TcR-stimulated phosphorylation of both proximal (for example, TcR-ζ chain) and distal (for example, ERK 1,2) signaling intermediates are inhibited in Herpes simplex virus-inactivated T cells, as is calcium flux. The signaling defect in inactivated cells was also found to be overcome by a combination of PMA and ionomycin which bypass the proximal block in the TcR pathway.

Experimental Procedures

Antibodies and Cytokines

Antibodies used were anti-CD3 (Clone OKT3) OrthoMcNeil, Redwood City, Calif.; anti-phosphotyrosine (clone 4G10), and anti-Lck, (clone 3A5), Upstate Biotechnology, Lake Placid, N.Y.; anti-Raf, (clone 53), BD Biosciences, San Diego, Calif.; anti-zeta chain, (clone 6B10), Biocarta, San Diego, Calif.; anti-HSP90α, (clone 9D2), Stressgen Biotechnologies, Victoria, Canada; rabbit-anti-phospho-ZAP70 (2701s), Cell Signaling, Beverly, Mass.; rabbit polyclonal anti-actin antibodies, Sigma, St. Louis, Mo. For flow cytometry, antibodies used were PerCp-conjugated anti-CD8 (clone SK1) or anti-CD4 (clone SK3), PE-conjugated anti-IFN-γ (clone 25723.11), and FITC-conjugated anti-TNF-α (clone 6401.1111), BD Pharmingen, Palo Alto, Calif. Secondary antibodies used were horse radish peroxidase-(HRP) conjugated goat-anti-mouse IgG or goat anti-rabbit IgG (Cell Signaling); rabbit anti-rat IgG (Stressgen); and donkey anti-rabbit FITC conjugated antibody (Jackson Immunologicals, Bar Harbor, Me.). Cytokines used were human recombinant interleukin (IL)-2 (Chiron, Emeryville, Calif.), IL-7 (RnDSystems, Minneapolis, Minn.) and IL-15 (Peprotech, Rocky Hill, N.J.).

Viruses

The following viruses are provided as models used in the described methods. Other viruses that posses the same characteristics defined herein are known and available to the skilled artisan and can be used in the methods described. HSV-1 (F), HSV-2 (HG52), R7041 (HSV-1 F with U_{S}3 deletion) and R7306 (R7041 with Us3 rescue) laboratory strains were grown from stocks originally obtained from B. Roizman (University of Chicago, Chicago, Ill.), tittered in Vero cells and used to infect fibroblasts as previously described in Example 1 and by Sloan et al. (J. Immunol. 171:6733, 2003). Cytomegalovirus (CMV) strains AD169 and Toledo were provided by M. Boeckh (Fred Hutchinson Cancer Research Center, Seattle, Wash.) and are also available from the ATCC. A varicella zoster virus (VZV) wild type clinical strain was provided by R. Morrow (University of Washington, Seattle, Wash.). Cells were infected with a multiplicity of infection (MOI) of 10 unless otherwise noted. LCMV (clone 13) virus was kindly provided by P. D. Greenberg (Fred Hutchinson Cancer Research Center, Seattle, Wash.). A recombinant vaccinia virus encoding the CMV immediate early protein 1 (Vac/IE1) under the p7.5 promoter, was provided by William Britt (University of Alabama, Birmingham, Ala.).

Cells

The primary human CD8 T lymphocyte clone, KSN is specific for the RVWDLPGVLK peptide (SEQ ID NO: 3) presented by HLA-A3 expressing targets. The SA-CD8 T cell clone, was provided by S. Riddell (Fred Hutchinson Cancer Research Center, Seattle, Wash.), recognizes the CMV immediate early protein 1 (IE1) restricted by HLA-B7. The primary CD4 T cell clone 9G8, was provided by J. McElrath (University of Washington, Seattle, Wash.) specifically recognizes a 20-mer peptide spanning amino acids 571-590 of the HIV-1_{MN} envelope protein and has demonstrated cytolytic activity against peptide coated cells restricted by MHC class II HLA-DR1 (Kent et al., J. Immunol. 158:807, 1997). T cell clones were maintained and propagated as described in Example 1. Immortalized (Jurkat E6.1) T cells were purchased from the American Type Culture Collection (ATCC, Manassas, Va.). Mouse CD8 T cells were isolated from B6; D2-TgN mice which express a transgene T cell receptor (TcR) specific for the LCMV peptide GP 33 AA33-41 presented by H-2D^{b}. To these cells, 50 U/mL recombinant human IL-2 (rhIL-2) and 1 μM LCMV peptide GP 33 AA3341 (C41M) (KAVYNFATC; SEQ ID NO: 4), was added to media every 3 days and T cell density was maintained at approximately 1×10⁶ cells/mL. Mouse 3T3 fibroblasts were provided by J. Vieira (University of Washington, Seattle, Wash.), Vero cells were purchased from the ATCC (ATCC CCL-81) and primary foreskin human fibroblasts were maintained in DMEM (Invitrogen) supplemented with 10% FCS and identical constituents described in Example 1. All cells were incubated at 37° C. in 5% CO₂.

T Cell Inactivation Assay

Fibroblasts were grown to confluency in flat bottom plates (Costar, Cambridge, Mass.) before infection with the viruses indicated or mock infected for 6 h unless otherwise noted. Acyclovir (50 μM) or ganciclovir (1 mg/mL) was added to media to prevent HSV maturation within fibroblasts and subsequent infection of lymphocytes, as described in Example 1 and Sloan et al. (J. Immunol. 171:6733, 2003). Following HSV infection of the [symbol] fibroblasts, the monolayer was washed with phosphate buffered saline (PBS) prior to co-incubation with T cells for 4 h. At the end of the incubation, T cells were gently aspirated off and counted. Fibroblasts were not removed with aspiration and this monolayer confluency was confirmed by visual inspection through the microscope. In assays comparing differences in signaling molecule phosphorylation states, T cells were serum starved for about 6 to about 10 h prior to co-incubation with fibroblasts.

Cytolytic T Cell Response

Cytolytic T cell response were assessed in triplicate by standard ⁵¹Cr-release assay, as described in Example 1 and Sloan et al. (J. Immunol. 171:6733, 2003). For SA-CMV-specific CD8 T cells, (lymphoblastoid cell lines) LCL targets were infected with Vac/IE for 4 hours while for 9G8-HIV-specific CD4 T cells, LCL targets were pulsed for 1 h with 1 μM of the HIV 20-mer peptide prior to being washed to remove excess peptide. MC57 cells were infected with LCMV at an MOI of 0.5 for 36 h prior to ⁵¹Cr labeling. Maximum spontaneous ⁵¹Cr release was always <20% of maximal release. Percentage of ⁵¹Cr release was calculated as follows:
((mean experimental cpm-mean spontaneous cpm)/(mean maximal cpm-mean spontaneous cpm))×100%.
Flow Cytometry Analysis of Cytokine Production and ERK Phosphorylation

For cytokine production analysis, T cells were stimulated with either plate-bound agonistic anti-CD3 antibody (clone OKT3) or 20 ng/mL phorbol ester myristate acetate (PMA, Sigma) plus 500 ng/mL ionomycin (Sigma) at 37° C. After 2 h of stimulation, 10 μg/mL brefeldin A (BD Biosciences) was added for a further 4 h to facilitate intracellular accumulation of cytokines. T cells were subsequently fixed, permeabilized and stained following the manufacturer's protocol (BD Pharmingen), and immediately analyzed on a FACSCalibur® flow cytometer (Becton Dickinson). Data were analyzed using CELLQUEST® software (Becton Dickinson). For assessment of the phosphorylation status of ERK in T cells, OKT3 (10 μg/ml) was used to trigger serum starved T lymphocytes for about 10 min on ice or at 37° C. Cells were then fixed with 2% paraformaldehyde for 10 min at 37° C. followed by the drop wise addition of ice-cold methanol with mixing and then [symbol] 30 min. Cells were then washed once with buffer (PBS 0.8% bovine serum albumin) followed by the addition of anti-phospho-ERK1/2 at a 1:100 dilution for 30 min at room temperature. Cells were washed once and a FITC-conjugated secondary antibody was added at 1:200 for 30 min at room temperature in the dark. Cells were washed once prior to analysis by flow cytometry. Normal rabbit serum used as a primary antibody negative control and resulted in no change in fluorescence intensity following anti-CD3 triggering.

Detection of Calcium Flux in T Cells

Mock- or HSV-infected T cells were incubated in the presence of 2 μM indo-1 (a calcium indicator available from Molecular Probes, Eugene, Oreg.) at 37° C. for 30 min with occasional agitation. Following two washes with cell loading medium (Hank Buffered Salt Solution (Invitrogen) containing 1 mM Ca⁺, 1 mM Mg²⁺ and 0.5% FCS), cells were allowed to rest for about 15 to about 30 min to allow for de-esterification of intracellular indo-1. Samples were then analyzed on a LSR1 flow cytometer (Becton Dickinson). After obtaining a baseline reading, cells were stimulated by the addition of OKT3 to a final concentration of 3 μg/mL and immediately placed back on the flow cytometer for analysis.

Western Blotting and Immunoprecipitation

Mock or inactivated T cells were used for western blotting or placed in ice water for about 10 to about 15 min prior to stimulation with 3 μg/mL OKT3 for various times as indicated at 37° C. Cells were then washed once in PBS and lysed for 30 min at 4° C. in lysis buffer (1% Nonidet P-40, 0.25% sodium deoxycholate, 50 mM Tris-HCl (pH 7.4), 150 mM NaCl, 1 mM EGTA, 1 mM NaF, 1 mM Na₃VO₄, 1 mM PMSF, 1 μg/mL TPCK, 1 μg/mL TLCK). Nuclei and cellular debris were removed by centrifugation for about 10 min at 10,000 rpm at 4° C. For evaluation of total protein tyrosine phosphorylation, cell lysates were boiled in denaturation buffer (55% sucrose, 20% SDS, 2 mg bromophenol blue, β-mercaptoethanol, 1 M Tris-HCl pH 7) for 5 min. For immunoprecipation assays, lysates were rotated overnight at 4° C. with immunoprecipitating antibody (1 μg/mL). Immune complexes were recovered by incubation with 40 μL of 50% packed protein-G agarose beads (Sigma) at 4° C. for about 1 to about 2 h. Pellets were washed with ice cold lysis buffer three times, re-suspended in SDS-PAGE loading buffer, and heated for 5 min at about 70 to about 90° C. Total cell lysates or immunoprecipitates were separated on 4 to 12% SDS-PAGE (Invitrogen) gradient gels and electrophoretically transferred onto nitrocellulose membrane [symbol] milk in PBS (blocking buffer), and probed with specific antibody overnight as per the manufacturer's directions. Membranes were then washed 3 times in PBS-0.1% Tween and 3 times in PBS followed by addition of horseradish peroxidase-linked secondary antibody (Cell Signaling) for about 1 to about 2 h and washed as per the manufacturer's directions. Bands of interest were visualized by enhanced chemiluminescent substrate (Pierce, Rockford, Ill.) as directed by the manufacturer. Re-probing of membranes with different primary antibodies was performed following 3 washes in PBS and membrane stripping (Pierce) as per the manufacturer's instructions.

Mass Spectrometry

Tyrosine phosphorylated proteins immunoprecipitated from control and HSV-inactivated T cells were separated on a 4 to 12% SDS-PAGE gel prior to Coomassie (Sigma) or silver staining (SILVERQUEST®, Invitrogen) as per the manufacturers' specifications. The 90 kD region was excised and subjected to proteolytic digestion with trypsin. Following digestion, samples were desalted using a microC18 ZipTip® (Millipore) and dried. Samples were then resuspended in 7 μL of 0.1% TFA and analyzed by LC/ESI MS/MS with LCQ DECA XP mass spectrometer (ThermoElectron) using an instrument configuration described by Gatlin, et al (Gatlin et al., Anal. Biochem. 263:93, 1998). Data were collected in a data dependent mode in which a MS scan was followed by MS/MS scans of the three most abundant ions from the preceding MS scan. Mass spectrometry data were searched against the human subset of the NCBI nonredundant protein database using the software search algorithm COMET (Institute for Systems Biology) (Shevchenko et al., Anal. Chem. 68:850, 1996). Protein identifications were considered valid if at least 2 peptides were identified back to a protein and if the peptide matches had raw scores of greater than 200 for +1 ions, 300 for +2 ions, and 300 for +3 ions, Z-scores greater than 4, and % ions of greater than 15%.

Results

HSV-Infected Fibroblasts Inactivate CD4 and CD8 T Lymphocytes, which are not of the T Regulatory Phenotype

KSN cells are a primary human CD8 T cell clone which recognize the RVWDLPGVLK peptide (SEQ ID NO: 3) presented by HLA-A3 expressing targets. The KSN cells were incubated with mock-, HSV1- or HSV2-infected fibroblasts in the presence of acyclovir. After 6 h, the KSN cells were gently aspirated, added to ⁵¹Cr-labeled target cells and the percent specific lysis was calculated at various effector:target cell ratios as described above. These data demonstrated that after incubation with mock-infected fibroblasts, the T cells were able to kill sensitive targets. However, these same T cells, after exposure to HSV-1 or -2 infected fibroblasts, had profoundly decreased cytolytic function compared to control T cells over a wide range of effector target ratios, a process termed T cell inactivation in Example 1 and Sloan et al. (J. Immunol. 171:6733, 2003). This lytic difference was not secondary to depletion of effector molecules within inactivated T cells due to killing of HSV-infected fibroblasts during co-culture, since no specific lysis by CTL was observed against mock- or HSV-infected fibroblasts. To determine if susceptibility to inactivation was limited to human CD8 T cells, an HIV-specific CD4 human T cell clone or LCMV-specific mouse CD8 T cells were incubated with mock- or HSV-infected fibroblasts and the ability of these T cells to kill target cells was tested. Both the human CD4 and mouse CD8 T cells were efficiently inactivated by HSV-infected fibroblasts as demonstrated by their reduced lytic activity. This suggested that HSV-infected cells were able to profoundly disarm various immune effector cells, both from humans and mice.

Many classic models of anergic T cells are reversible by addition of IL-2 (Schwartz, Curr. Biol. 10:R-572, 2003). In Example 1 it was previously demonstrated that IL-2 was unable to restore T cell lytic function in HSV-inactivated T cells (See above and in Sloan et al., J. Immunol. 171:6733, 2003). Similarly in this example, treatment with purified IL-7 or IL-15 in short-term cultures was found not to restore lytic function to HSV-inactivated T cells. To determine whether inactivation of T cells involved the generation of a regulatory T cell population (Shevach, 2000; Shevach et al., Adv. Exp. Med. Biol. 490:21, 2001), HSV-inactivated T cells were mixed with control T cells at a ratio of 1:1 and their lytic capacity measured by chromium release. No observed significant down-regulation of lytic activity by either CD4 or CD8 T cells following this co-culture was noted, suggesting that inactivated T cells in this model were not of the T regulatory phenotype.

VZV and CMV do not Inactivate T Cells

In order to determine if this T cell inactivation could be mediated by fibroblasts infected with other closely related herpes viruses, CD8 T cells were incubated with mock-infected fibroblasts or with either VZV- or CMV-infected fibroblasts and chromium release was measured. T cells cultured with VZV- or CMV-infected fibroblasts killed target cells at levels similar to control T cells, indicating that these other herpes viruses were not able to inactivate T cells. To determine if this observation was due to a low viral [symbol] case of VZV or slower growth kinetics in the case of CMV, a 5-fold higher MOI of VZV (MOI of 50) and an 18 h fibroblast infection with CMV was used. Furthermore, since there are many CMV laboratory variants, two different strains were used. Infection of the fibroblasts by these viruses was confirmed by the appearance of widespread and diffuse cytopathic effect. T cell inactivation was not observed under any of these conditions, indicating that this effect was specific for HSV.

Lymphocytes Incubated with HSV-Infected Fibroblasts Undergo Tyrosine Phosphorylation of 50 and 90 kD Proteins

T cell function is regulated in part by phosphorylation and dephosphorylation of numerous intracellular mediators. To determine if inactivation by HSV was due to differences in phosphorylation patterns, total tyrosine phosphorylated proteins were compared between control and inactivated lymphocytes by Western blot analysis. Briefly, lymphocytes were exposed to fibroblasts infected with the viruses discussed below, or mock-infected fibroblasts. Two strongly phosphorylated bands were observed at 50 and 90 kD in the HSV-inactivated cells but not control cells. In Example 1 it was demonstrated that HSV lacking the Us3 gene was unable to inactivate T cells, as was UV-inactivated HSV (which was unable to initiate viral DNA transcription and subsequent de novo viral protein synthesis). To verify that these phosphorylated bands were specific to HSV-inactivated T cells and were not the result of culture conditions, cell lysates from T cells were exposed to mock-, Us3 deletion mutant HSV-, or UV-inactivated HSV-infected fibroblasts. As controls, U_{S}3 rescue mutant HSV- and wild-type HSV1 (F)- and HSV2-infected fibroblasts were simultaneously compared. The 90 and 50 kD phosphorylated bands were not observed in T cells exposed to mock-infected fibroblasts, fibroblasts infected with HSV lacking the Us3 gene, or fibroblasts infected with UV-inactivated HSV. However, these bands were readily visible in T cells exposed to fibroblasts infected with wild type HSV1, HSV2, or the Us3 rescue HSV-mutant. Similarly, T cells exposed to VZV or CMV-infected fibroblasts did not display phosphorylated 90 and 50 kD bands. Interestingly, EBV-immortalized B lymphocytes similarly exposed to HSV-infected fibroblasts, but not mock-infected fibroblasts, displayed the same phosphorylated 90 kD and 50 kD bands. These observations demonstrate that for HSV-infected fibroblasts to cause phosphorylation of the 50 and 90 kD proteins in T cells and thus inactivate them, synthesis of viral proteins within the fibroblasts was necessary and that in particular Us3, as HSV early gene encoding a serine/threonine kinase, was required.

Because the 90 kD band was highly abundant in the Western blots, determination the identity of this phosphorylated protein was sought. Tyrosine phosphorylated proteins from control and HSV-inactivated T cells were immunoprecipitated and separated by SDS-PAGE gel electrophoresis. The region of the gel containing the 90 kD band was subjected to mass spectrometry analysis and comparison of ionization fragments to the NCBI database provided a match to heat shock protein 90 (HSP90) from HSV-inactivated T cells, but not from control T cells. To confirm the phosphorylation of HSP90, immunoprecipitation was performed on lysates from control or HSV-inactivated T cells using antibodies recognizing HSP90 or total phosphotyrosine. The immunoprecipitates were then probed with anti-phosphotyrosine or anti-HSP90 antibody. HSP90 was immunoprecipitated from inactivated T cells reacted with anti-phosphotyrosine, while immunoprecipitated HSP90 from control cells was not. In addition, immunoprecipitated total phosphotyrosine from HSV-inactivated T cell extract reacted with anti-HSP90 antibody, while immunoprecipitated total phosphotyrosine from control cell extract did not. These findings suggest that phosphorylation of HSP90 was likely to be involved in the inactivation of T cells by HSV.

Inactivated T Cells have Disrupted Interactions Between Lck, Raf and HSP90

HSP90 is a chaperone protein required as a scaffold for proper function of several proteins involved in TcR signaling, including p56^{lck} and Raf (Giannini and Bijlmakers, Mol. Cell. Biol. 24:5667, 2004; Schulte et al., J. Biol. Chem. 270:24585, 1995; Xu et al., J. Cell. Physiol. 198:188, 2004). p56^{lck} is known to be associated with TcR components and becomes phosphorylated rapidly after TcR ligation (Hartson et al., Biochem. 35:13451, 1996). Raf is a kinase that is distal from the TcR in the signaling cascade and is necessary for activation of MAP kinases required for full T cell function (Schulte et al., J. Biol. Chem. 270:24585, 1996). Studies using geldanamycin, which blocks the ATP binding site of HSP90, have shown that it disrupts the association between p56^{lck} and Raf with HSP90 within T lymphocytes (Yorgin et al., J. Immunol. 164:2915, 2000). Furthermore, this association was shown to be required for proper function of these client proteins and T cell function (Piatelli et al., J. Biol. Chem. 277:12144, 2002). Therefore, the association of HSP90 with p56^{lck} and Raf in HSV-inactivated T cells was evaluated. Briefly, cell extracts from T cells exposed to mock or HSV-2 infected fibroblasts were immunoprecipitated with antibodies specific for HSP90, Raf, or Lck, and subjected to immunoblot analysis with antibodies specific for HSP90. Results were determined from a single blot representative of six independent experiments. As expected, in control T cells p56^{lck} and Raf were constitutively associated with HSP90, as evidenced by their co-immunoprecipitation from mock infected cells. However, in HSV-inactivated T cells HSP90 no longer co-immunoprecipitated with p56^{lck} or Raf. Thus, tyrosine phosphorylation of HSP90 in HSV-exposed T cells resulted in disruption of binding to client signaling proteins, providing a potential explanation for lymphocyte inactivation by HSV infected fibroblasts.

Multiple Signal Transduction Pathways are Disrupted in Inactivated T Cells

To determine if ablation of HSP90 binding to p56^{lck} resulted in a block in subsequent signaling in HSV-inactivated T cells and thus might be a key event in mediating this phenotype, phosphorylation of the TCR-ζ chain (which is phosphorylated by p56^{Lck}) and ZAP-70 (which occurs subsequent to phosphorylation of the TCR-ζ chain) was examined. Briefly, cell extracts from T cells exposed to mock- or HSV-infected fibroblasts were immunoprecipitated with anti-phosphotyrosine antibody 4G10 or anti-TcR-ζ antibody, stimulated with soluble anti-CD3 antibody for variable times, and subjected to Western immunoblot analysis with antibodies specific for TcR-ζ, phosphotyrosine, or ZAP-70. After stimulation with the agonistic antibody OKT3, the TCR-ζ chain in control cells showed an increase in the hyperphosphorylated form, while in HSV-inactivated T cells, the TCR-ζ chain remained hypo-phosphorylated. ZAP-70 also failed to become phosphorylated within OKT3-stimulated HSV-inactivated T cells compared to controls, further supporting the notion that p56^{lck} dysfunction was secondary to HSP90 phosphorylation.

ZAP-70 phosphorylation and subsequent activation of linker of T cells (LAT) is required for phosphorylation of downstream kinases as well as intracellular Ca²⁺ signaling. In order to examine the effects of HSP90 phosphorylation on downstream signaling intermediates within stimulated HSV-inactivated T cells, ERK phosphorylation (activation) was evaluated by flow cytometry. ERK is a distal signaling intermediate in the mitogen activated protein kinase (MAPK) signaling cascade (Piatelli et al., J. Biol. Chem. 277:12144, 2002). Impaired ERK phosphorylation, as determined by flow cytometric analysis using antibody specific for the phosphorylated form of ERK1, was observed in HSV-inactivated T cells after stimulation with OKT3 (10 min), compared to control T cells. In order to determine if the block in inactivated T cells affected Ca²⁺ signaling, Ca²⁺ flux, as determined by radiometric flow cytometry analysis using the calcium-sensitive probe indo-1, was assessed in soluble anti-CD3 antibody (OKT3) stimulated control and HSV-inactivated T [symbol] cells. In these studies the Ca²⁺ flux was also observed to be profoundly impaired within HSV-inactivated T cells.

The blockade of p56^{lck} phosphorylation of the TCR-ζ chain would be consistent with the observed signaling defects in inactivated T cells. If this were indeed the main defect in inactivated T cells, it would be predict that they should retain the ability to respond to stimuli acting downstream of this block. To test this hypothesis, mock or inactivated T cells (exposed to HSV-infected fibroblasts) were stimulated with the agonistic soluble anti-CD3 antibody OKT3, or with a combination of PMA plus ionomycin. PMA directly activates protein kinase C, while ionomycin leads to mobilization of intracellular calcium. Both these agents act downstream of the proposed block of p56^{Lck} activity. As predicted, while inactivated T cells failed to synthesize TNF-α and IFN-γ after OKT3 stimulation, they responded vigorously to PMA and ionomycin.

Discussion

In example 1 it was demonstrated that T cell contact with HSV-infected fibroblasts results in a dysfunctional state that is characterized by inhibition of cytokine production and cytolysis (Sloan et al., J. Immunol. 171:6733, 2003). The aim of the studies described in this current example was to define the underlying mechanism responsible for causing this dysfunctional state within lymphocytes. The data from this example demonstrate that the HSV-infected fibroblast, after finite contact with a lymphocyte, altered lymphocyte physiology by causing tyrosine phosphorylation of HSP90. HSP90 is required for proper folding and function of several client proteins, including Raf and p56^{Lck}, two critical signaling intermediates necessary for T cell activation (Yorgin et al., J. Immunol. 164:2915, 2000), and the data discussed above demonstrated that HSP90 phosphorylation prevented its binding to Raf and p56^{Lck}. In addition, it was demonstrated that the immediate downstream substrate of p56^{Lck}, the TCR-ζ chain, was hypophosphorylated when HSV-inactivated T cells were triggered through the TCR using agonistic anti-CD3 antibody. ZAP-70, immediately downstream from TCR-ζ, also failed to become phosphorylated following TCR stimulation of inactivated cells. Furthermore, it was demonstrated that Ca²⁺ flux, which is dependent on successful proximal TCR signaling, was profoundly inhibited in HSV-inactivated T cells. This inhibition also extended to distal signaling intermediates, as demonstrated by the failure of ERK to become phosphorylated in OKT3 stimulated HSV-inactivated T cells. Consistent [symbol] cells responded normally to stimulation with a combination of PMA and ionomycin, which bypasses the block in the proximal TCR signaling pathway.

Previous reports have implicated a critical role for HSP90 in the function of the TCR signaling pathway (Sato et al., Proc. Natl. Acad. Sci. USA 97:10832, 2000); Schnaider et al., Life Sci. 63:949, 1998). HSP90 interacts with and stabilizes p56^{lck} and Raf, enzymes necessary for successful T lymphocyte activation through the TCR. The interaction between HSP90 and these client proteins can be disrupted by geldanamycin, a compound that blocks the ATP binding site of HSP90 (Schnaider et al., supra). The disruption of the association between p56^{lck} and Raf to HSP90 within T lymphocytes has been shown to result in the failure of T cells to respond to stimulation with anti-CD3 (Schnaider et al., supra) or anti-CD3 plus anti-CD28 (Yorgin et al., J. Immunol. 164:2915, 2000). The above findings with HSV-inactivated T cells demonstrated that the phosphorylation of HSP90 resulted in an analogous disruption of the interaction of HSP90 with p56^{Lck} and Raf, with similar disruption of the TCR signaling pathway.

Several groups have reported that phosphorylation of HSP90 represents a mechanism for control of chaperone-client protein interactions and cellular signaling. Zhao et al. have shown that serine/threonine phosphorylation regulates attachment of HSP90 to the reovirus attachment protein σ1 (Zhao et al., J. Biol. Chem. 276:32822, 2001). Tyrosine phosphorylation of HSP90 has been reported to occur during mammalian sperm capacitation (Ecroyd et al., Biol. Reprod. 69:1801, 2003; Ficarro et al., J. Biol. Chem. 278:11579, 2003). In endothelial cells, HSP90 is tyrosine phosphorylated after exposure to bradykinin (Harris et al., Gen. Pharmacol. 35:165, 2000) or atorvastatin (Brouet et al., Circ. Res. 89:866, 2001). Perhaps the best analogy to the system described herein is the purinergic P2X7 receptor-protein complex, of which HSP90 is a component. Selective tyrosine phosphorylation of P2X7 receptor-associated HSP90 leads to negative regulation of P2X7 receptor complex formation and function (Adinolfi et al., J. Biol. Chem. 278:37344, 2003).

One caution that should be mentioned is that it remains possible, although unlikely, that in the inactivated T cell model it is not HSP90 itself that is phosphorylated, but rather another unidentified 90 kD protein that co-immunoprecipitates with HSP90. To date direct identification of a site of tyrosine phosphorylation of HSP90 by mass spectrometry has been unsuccessful, and other workers have been unsuccessful as well (Ficarro et al., J. Biol. Chem. 278:11579, 2003). A similar note of caution was raised by Adinolfi et al, in the P2X7 model [symbol] (Adinolfi et al., J. Biol. Chem. 278:37344, 2003), and this caveat is applicable to most other reports of HSP90 phosphorylation. However, Ecroyd et al demonstrated tyrosine phosphorylation of HSP90 by 2D gel electrophoresis (Ecroyd et al., Biol. Reprod. 69:1801, 2003), and it is highly unlikely that the putative co-immunoprecipitating protein would be identical to HSP90 in terms of both molecular weight and isoelectric point.

The relationship of the T cell inactivation described herein to the phenomenon of anergy is of interest. Although the anergy literature is complex, one useful conceptual framework is that of adaptive tolerance (Migita et al., J. Immunol. 155:5083, 1995; Schwartz, Curr. Biol. 10:R572, 2003; Utting et al., J. Immunol. 164:2881, 2000). The described HSV-inactivated T cells have similarities and differences with this anergy model. Briefly, in adaptive T cell tolerance, there is generalized inhibition of effector functions in naïve T cells. The defect is due to an early block in tyrosine kinase activation (failure to phosphorylate TCR-ζ and subsequently ZAP-70) that is associated with a profound inhibition in calcium mobilization, and which is not reversed after the addition of IL-2. HSV-inactivated T cells, due to their disrupted HSP90-p56^{Lck} association, are similar to T cells in the adaptive anergy model in that they have an early block in tyrosine kinase activation. This in turn inhibits calcium mobilization and phosphorylation of downstream kinases, and thus HSV-inactivated T cells are unresponsive to IL-2 (and IL-7, IL-15). Unlike the adaptive tolerance model, however, the T cells used in the above inactivation experiments were not naïve T lymphocytes. Furthermore, unlike most anergy models, inactivation appears to be induced without involvement of the TcR, since the HSV-infected fibroblasts used in the present system do not express the antigens recognized by the T cells to be inactivated.

T lymphocytes play a central role in the control of many different viral infections including herpesvirus infection in humans (Oxenius et al., Immunity 9:449, 1998; Zajac et al., J. Exp. Med. 188:2205, 1998). The continuous interaction between host T cells and different viruses during their co-evolution has shaped not only the ability of the immune system to respond to these pathogens, but also the counter measures used by viruses to evade the immune system (Zinkemagel, Science 271:173, 1996). In summary, HSV, but not other related herpes viruses such as VZV and CMV, has found a way to induce a profound dysfunctional state in T cells after contact with HSV-infected cells by phosphorylating HSP90 and disrupting its interaction with client proteins necessary for T cell function. A detailed understanding of the interactions of HSV with the immune system may allow development of more adequate vaccine strategies against this virus. Furthermore, the disclosed model may also provide a simple and robust model for study of biochemical events associated with T cell regulation and dysfunction and can be used for the identification of agents that will prevent T cell inactivation in HSV infection.

The previous examples are provided to illustrate but not to limit the scope of the claimed inventions. Other variants of the inventions will be readily apparent to those of ordinary skill in the art and encompassed by the appended claims. All publications, patents, patent applications, and other references cited herein are hereby incorporated by reference.

What is claimed is:

1. A method of screening an agent for activity in modulating T lymphocyte function, the method comprising:
(1) contacting the agent with
(a) a cell expressing a HSV U_{S}3 polypeptide and one or more other HSV proteins; and/or
(b) a T lymphocyte;
(2) contacting the HSV U_{S}3-expressing cell with the T lymphocyte;
(3) contacting a second HSV U_{S}3-expressing cell with a second T lymphocyte, wherein the second HSV U_{S}3-expressing cell and the second T lymphocyte are not contacted with the agent;
(4) determining for each of the first and second T lymphocyte the level of a physiological change associated with T lymphocyte function; and
(5) comparing the relative levels of the physiological change determined for each of the first and second T lymphocytes to determine whether the agent modulates T lymphocyte function.

2. The method of claim 1, further comprising contacting the first and second T lymphocytes with a second agent that activates the T cell receptor (TcR-activating agent).

3. The method of claim 1, wherein the cell expressing the HSV U_{S}3 polypeptide and late HSV proteins is infected with herpes simplex virus (HSV).

4. The method of claim 3, wherein the HSV is HSV-1 or HSV-2.

5. The method of claim 1, wherein the cell expressing the HSV U_{S}3 polypeptide and HSV proteins is a recombinant cell.

6. The method of claim 1, wherein the HSV U_{S}3 polypeptide has an amino acid sequence that has 90% sequence identity with the sequence set forth in SEQ ID NO:1 or SEQ ID NO:2.

7. The method of claim 6, wherein the HSV U_{S}3 polypeptide has the amino acid sequence set forth in SEQ ID NO:1 or SEQ ID NO: [symbol] .

8. The method of claim 1, wherein the cell expressing the HSV U_{S}3 polypeptide is a fibroblast.

9. The method of claim 1, wherein the physiological change is production of a cytokine.

10. The method of claim 9, wherein the cytokine is selected from the group consisting of IFN-γ and TNF-α.

11. The method of claim 1, wherein the physiological change is exocytosis of lytic granules from the T lymphocyte.

12. The method of claim 1, wherein the physiological change is a change in the relative phosphorylation level of a cellular protein.

13. The method of claim 12, wherein the cellular protein is a cytotoxic T lymphocyte (CTL) protein.

14. The method of claim 13, wherein the CTL cellular protein is associated with the TcR or the TcR signaling cascade.

15. The method of claim 13, wherein the CTL cellular protein is heat shock protein 90 (HSP90) or a 50 kD protein.

16. The method of claim 15, wherein the phosphorylation of HSP90 and/or the 50 kD protein is increased.

17. The method of claim 15, wherein the phosphorylation of HSP90 and/or the 50 kD CTL protein is decreased.

18. The method of claim 2, wherein the TcR-activating agent is staphylococcal enterotoxin B (SEB).

19. The method of claim 2, wherein the TcR-activating agent is an anti-CD3 antibody.

20. The method of claim 19, wherein the anti-CD3 antibody is immobilized on a solid phase.

21. The method of claim 19, further comprising contacting the T lymphocyte with a target cell expressing Fcγ receptor.

22. The method of claim 21, wherein the Fcγ receptor-expressing cell is a FcγR⁺ P815 target cell.

23. The method of claim 2, wherein the TcR-activating agent is a target antigen bound to an MHC class I molecule on the surface of a target cell, and wherein the first and second T lymphocyte specifically recognize the target antigen.

24. The method of claim 23, wherein the target cell is an EBV-transformed B cell line.

25. The method of claim 23, wherein the HSV U_{S}3-expressing cell and the target cell are the same cell.

26. The method of claim 23, wherein the physiological change is associated with apoptosis of the target cell.

27. The method of claim 26, wherein the apoptosis-associated physiological change is lysis of the target cell.

28. The method of claim 27, wherein the target cell is ⁵¹Cr-labeled and lysis of the target cell is determined by detecting release of the ⁵¹Cr from the target cell.

29. The method of claim 26, wherein the apoptosis associated physiological change is activation of a caspase.

30. The method of claim 29, wherein the caspase is caspase 3.

31. A method of screening an agent for activity in modulating cytotoxic T lymphocyte (CTL) function, the method comprising:
(1) contacting the agent with
(a) a fibroblast infected with HSV; and/or
(b) a CTL;
(2) contacting the HSV-infected fibroblast with the CTL;
(3) contacting a second HSV-infected fibroblast with a second CTL, wherein the second HSV-infected fibroblast and the second CTL are not contacted with the agent;
(4) determining for each of the first and second CTLs the level of a physiological change associated with CTL function; and
(5) comparing the relative levels of the physiological change determined for each of the first and second CTLs is to determine whether the agent modulates CTL function.

32. The method of claim 31, wherein the physiological change determined for each of the first and second CTLs is to determine the relative level of phosphorylation of heat shock protein 90 (HSP90) and/or a 50 kD CTL protein, and selecting the agent that reduces or inhibits the relative increase in the level of phosphorylation.

33. The method of claim 32, wherein the fibroblast is infected with HSV-1 or HSV-2.

34. A method for blocking suppression of cytotoxic T cell (CTL) activity against HSV-infected target cells comprising:
blocking the expression or functional activity of HSV U_{S}3.

35. A method for suppressing cytotoxic T cell (CTL) activity against a target antigen in a subject, the method comprising:
isolating a population of antigen presenting cells (APCs) presenting the target antigen on the cell surface;
introducing into the APCs an expression vector encoding an HSV U_{S}3 polypeptide and one or more other HSV proteins, whereby the APCs express the HSV U_{S}3 polypeptide and one or more other HSV proteins; and
administering to the subject the APCs expressing to the HSV U_{S}3 polypeptide and one or more other HSV proteins, thereby suppressing CTL activity against the target antigen in the subject.

36. A method of screening an agent for activity in suppressing T lymphocyte function, the method comprising:
(1) contacting the agent with a T lymphocyte;
(2) determining the level of phosphorylation of HSP90 and/or a 50 kD T lymphocyte protein;
(3) comparing the level of phosphorylation of HSP90 and/or the 50 kD T lymphocyte protein in the T lymphocyte contacted with the agent to a T lymphocyte that has not been contacted with the agent; and
(4) selecting the agent that increases the level of phosphorylation of HSP90 and/or 50 kD T lymphocyte protein for suppressing T lymphocyte function.


SEQUENCE LISTING (4 sequences)

SEQ ID NO: 1 - protein, 481 aa, Herpes simplex virus 1
MACRKFCRVYGGQGRRKEEAVPPETKPSRVFPHGPFYTPAEDACLDSPPPETPKPSHTTPPSEAERLCHLQEILAQMYGNQDYPIEDDPSADAADDVDEDAPDDVAYPEEYAEELFLPGDATGPLIGANDHIPPPCGASPPGIRRRSRDEIGATGFTAEELDAMDREAARAISRGGKPPSTMAKLVTGMGFTIHGALTPGSEGCVFDSSHPDYPQRVIVKAGWYTSTSHEARLLRRLDHPAILPLLDLHVVSGVTCLVLPKYQADLYTYLSRRLNPLGRPQIAAVSRQLLSAVDYIHRQGIIHRDIKTENIFINTPEDICLGDFGAACFVQGSRSSPFPYGIAGTIDTNAPEVLAGDPYTTTVDIWSAGLVIFETAVHNASLFSAPRGPKRGPCDSQITRIIRQAQVHVDEFSPHPESRLTSRYRSRAAGNNRPPYTRPAWTRYYKMDIDVEYLVCKALTFDGALRPSAAELLCLPLFQQK

SEQ ID NO: 2 - protein, 481 aa, Herpes simplex virus 2
MACRKFCGVYRRPDKRQEASVPPETNTAPAFPASTFYTPAEDAYLAPGPPETIHPSRPPSPGEAARLCQLQEILAQMHSDEDYPIVDAAGAEEEDEADDDAPDDVAYPEDYAEGRFLSMVSAAPLPGASGHPPVPGRAAPPDVRTCDTGKVGATGFTPEELDTMDREALRAISRGCKPPSTLAKLVTGLGFAIHGALIPGSEGCVFDSSHPNYPHRVIVKAGWYASTSHEARLLRRLNHPAILPLLDLHVVSGVTCLVLPKYHCDLYTYLSKRPSPLGHLQITAVSRQLLSAIDYVHCKGIIHRDIKTENIFINTPENICLGDFGAACFVRGCRSSPFHYGIAGTIDTNAPEVLAGDPYTQVIDIWSAGLVIFETAVHTASLFSAPRDPERRPCDNQIARIIRQAQVHVDEFPTHAESRLTAHYRSRAAGNNRPAWTRPAWTRYYKIHTDVEYLICKALTFDAALRPSAAELLRLPLFHPK

SEQ ID NO: 3 - protein, 10 aa, Homo sapiens
RVWDLPGVLK

SEQ ID NO: 4 - protein, 9 aa, Lymphocytic choriomeningitis virus
KAVYNFATC